(19) United States
(12) Patent Application Publication
(10) Pub. No.: US 2018/0105834 A1
(43) Pub. Date: Apr. 19, 2018

(54) A METHOD OF SITE-DIRECTED INSERTION TO H11 LOCUS IN PIGS BY USING SITE-DIRECTED CUTTING SYSTEM

(71) Applicant: INSTITUTE OF ANIMAL SCIENCES, CHINESE ACADEMY OF AGRIGULTURAL SCIENCES, Beijing (CN)

(72) Inventors: Kui Li, Beijing (CN); Jinxue Ruan, Beijing (CN); Shulin Yang, Beijing (CN); Yulian Mu, Beijing (CN); Hegang Li, Beijing (CN); Tianwen Wu, Beijing (CN); Jingliang Wei, Beijing (CN); Kui Xu, Beijing (CN); Lei Huang, Beijing (CN); Rong Zhou, Beijing (CN); Nan Liu, Beijing (CN)

(21) Appl. No.: 15/531,717

(22) PCT Filed: Nov. 27, 2014
(86) PCT No.: PCT/CN2014/092321

§ 371 Date: May 30, 2017

(51) Int. Cl. C12N 15/85; C12N 15/11; C40B 50/06; A01K 67/027

(52) U.S. Cl. CPC C12N 15/8509; C12N 15/11; C40B 50/06; A01K 67/0275; A01K 2217/05; C12N 2310/20; A01K 2227/108; A01K 2267/03; C12N 2015/8527; C12N 2800/30; C12N 2800/90; A01K 2217/07

(57) ABSTRACT

The present invention provides a method of site-directed insertion to H11 locus in pigs by using site-directed cutting system, includes the following steps: 1) identify the targeted sequence targeted by the targeted cutting system in the targeted genome sequence of pigs; 2) design and construct the targeting sequence of the corresponding cutting system according to the targeted site; 3) construction of targeting vector; 4) transfect cells, identify the efficiency of fixed-point insertion by PCR amplification. The invention is dependent on the site-directed cutting system of H11 locus in pigs, to insert the target gene into the target site, in order to solve the problems such as low efficiency of traditional shooting technique, inconvenience design of PCR detection primer, harder to detect. The invention provides a method of site-directed insertion which can stably express the foreign gene at the H11 locus, to build an efficient platform for the production of transgenic pigs.

### TECHNICAL FIELD

The present invention belongs to the field of genetic engineering, in particular to a method of site-directed insertion to H11 locus in pigs by using site-directed cutting system.

### BACKGROUND ART

Known in biotechnology research, the target gene is inserted into the genome of the chromosome homologous by using the methods of homologous recombination or transposons, but the practice shows that the low efficiency of homologous recombination, difficult operation, and the original gene was destroyed because of the insertion of the target gene; using the method of transposons, there are some problems such as the site of insertion into the chromosome is random, and the transposase is expensive.

Therefore, due to the limitations of the use of these technologies, in the cultivation of improved varieties of pigs, the foreign genes are randomly inserted into the genome of pigs, thereby the obtained recombinant by using of the corresponding technology make the subsequent breeding and phenotypic analysis very cumbersome.

In 2010, Simon Hippenmeyer of Stanford University and his research team isolated and identified a good gene insertion site on the chromosome 11 in mice, named hipp11 sites, referred to as H11 locus. The H11 site is located in the gap between the two genes Eif4enif1 and Drg1, adjacent to the exon 19 of Eif4enif1 gene and the exon 9 of Drg1 gene, the size of about 5 kb. Because the H11 locus is located between the two genes, it has high security, no gene silencing effect, and has broad-spectrum activity of cell expression. Experiments confirmed that there is no difference in the growth and development between the wild-type mice and the mice modified by Hipp11 site-directed gene. Currently there is similar Ros26 locus, but the site is a gene whose promoter is a broad-spectrum systemic expression, difficult to achieve tissue-specific expression, however there are no similar difficulties in H11 sites, because it is located between two genes, and promoters do not exist, so you can select the desired test promoter to complete spatio-temporal-specific expression of a gene of interest, the better to achieve mission objectives. If the safe and effective genetic modification site such as hipp11 is located in the genome of the pig, it will be conducive to the stability of transgenic pig breeding technology system.

The main method developed in recent years is the precise gene modification based on the sequence-specific nuclease. The sequence-specific nuclease is mainly composed of a DNA recognition domain and an endonuclease domain capable of nonspecific cleavage DNA. The main principle is that the DNA recognition domain firstly recognizes and binds to the DNA fragment needed to transform, then the DNA is cutted by the non-specific enzyme structure connected with DNA, cause the Double-strand break (DSB) of the DNA, the DSB activates DNA's self repair and causes mutations of gene to promote homologous recombination at the site.

ZFN and TALEN targeting technology is two more mature site-directed mutagenesis techniques in the present study, Zinc finger nuclease technology (Zinc Finger Nuclease, ZFN) is the gene precise modification techniques as mentioned in the preceding paragraph, composed of a specific DNA recognition domain and a non-specific endonuclease. In the ZFN recognition domain, a zinc finger structure may specifically identify plurality (typically three) consecutive bases, and the plurality zinc finger could recognize a series of bases. Therefore, in the design process of ZFN, the amino acid sequence of the zinc finger recognition domain is the focus, in particular, how to design more lysine2-histidine 2 (Cys2-His2) zinc finger protein in series, and how to decide the specific nucleotide triplet identified by each zinc finger protein by altering the 16 amino acid residues of α-helix.

The feasibility of ZFN technology in gene targeting modification has made it widely used in the gene modification of individual level and cellular level. First of all, it was realized by using of ZFN technology to achieve the gene targeted modification of the cellular level. For example, company Sangamo for the first time achieved ZFN mediated gene targeting in cultured human cell lines in 2005, and achieved the targeted gene site insertion through homologous recombination genes by using the same ZFN in 2007. Recently, people used ZFN to achieve the targeted mutation of the gene in human iPS and ES cells.

In contrast, the transcription activator-like effector nucleases (TALEN) has more advantages, it is another new technology which can achieve efficient site directed modification of the genome following the zinc finger nuclease technology. Transcription factor activation effector family has a protein (TALEs) which can identify and combine DNA. The specific binding of TALE and DNA sequence is mainly mediated by 34 constant amino acid sequences in TAL structure. The TALEs is connected with the cutting domain of FokI endonuclease, to form the TALEN, so that the double chain of the genome DNA can be modified at the specific sites.

There is a repeating area in the center of the TALE, which is usually made up of the repeating units with a variable number of 33-35 amino acids. Repeat Domain is responsible for identifying the specific DNA sequences. Each repeat sequence is essentially the same, except for the two variable amino acids, that is Repeat-Variable Diresidues (RVD). DNA recognition mechanism of TALE is that the RVD on a repeat sequence can identify a nucleotide on the DNA target point, and then fuse FokI nucleic acid enzyme, to combine into TALEN. TALEN is a heterodimer molecule (TALE DNA-binding domain of the two units are fused to the catalytic domain of one unit), can cut two sequences which are close to each one, making specific enhancements, so that the specificity is enhanced. The enzyme has the advantages of high efficiency, low toxicity, short preparation period, low cost and so on, that become increasingly evident.

(CRISPR)/CRISPR-associated (Cas) is a kind of evolving immune defense mechanism of the bacteria and the ancient bacteria. In recent years, researchers found that CRISPR/Cas9 use a small RNA to recognize and cut DNA to degrade foreign nucleic acid molecule. Cong etc. and Mali etc. can also prove that the Cas9 system can carry out effective targeted enzyme digestion in 293T, K562, iPS cells and other kinds of cells, and the efficiency of non-homologous recombination (NHEJ), homologous recombination (HR) is 3-25%, equivalent to the efficiency of the TALEN enzyme digestion. They also demonstrated that multiple targets can be simultaneously carried out targeted enzyme digestion.

The efficiency of traditional targeting is very low, which is completed mainly dependent on random exchange of intracellular homologous recombinant, the efficiency is very low. With the help of the above mentioned target cutting techniques, it will provide a good support for the research of gene function and breeding of animals and plants.

### DISCLOSURE OF THE INVENTION

An object of the present invention is to provide a method of site-directed insertion to H11 locus in pigs by using site-directed cutting system in order to solve the defects of the present technique, such as random insertion, complicated steps, expensive price and so on.

To achieve the above purpose, method provided by the invention includes the following steps: 1) identify the targeted sequence targeted by the targeted cutting system in the targeted genome sequence of pigs; 2) design and construct the targeting sequence of the corresponding cutting system according to the targeted site; 3) construction of targeting vector; 4) transfect cells, identify insert results by PCR amplification.

Wherein said targeted cutting system in step 1) is a TALEN targeted cutting system or CRISPR/Cas targeted cutting system.

Wherein said nucleotide cleaving enzyme using in CRISPR/Cas target cutting system is csa9 or cas9n.

Wherein said targeted sequence targeted by the targeted cutting system in step 1) is the targeted sequence targeted by TALEN, CRISPR/Cas9 targeted cutting system or targeted sequence targeted by CRISPR/Cas9n targeted cutting system.

Wherein said targeted sequences in step 1) are shown in 1), 2) or 3):

1) The targeted sequences targeted by TALEN targeted cutting system are a pair of sites, having nucleotide sequences shown in SEQ ID NO:1 and SEQ ID NO:4, SEQ ID NO:2 and SEQ ID NO:4, SEQ ID NO:3 and SEQ ID NO:4, SEQ ID NO:1 and SEQ ID NO:5, SEQ ID NO:2 and SEQ ID NO:5, or SEQ ID NO:3 and SEQ ID NO:5;

2) The targeted sequences targeted by CRISPR/Cas9 targeted cutting system are shown in SEQ ID NO:6 or SEQ ID NO:7.

3) The targeted sequences targeted by CRISPR/Cas9n targeted cutting system is a pair of sites, having nucleotide sequences shown in SEQ ID NO:8 and SEQ ID NO:9.

Wherein said targeted sequences in step 2 are polypeptide sequences of TALEN targeted cutting system, nucleotide sequences of CRISPR/Cas9 targeted cutting system or a pair of nucleotide sequences of CRISPR/Cas9n targeted cutting system.

Wherein said the polypeptide sequences of TALEN targeted cutting system include polypeptide A and polypeptide B, the specific sequences are shown in 1), 2), 3), 4), 5) or 6):

1) The specific sequences of the polypeptide A are shown in SEQ ID NO:10, specific sequences of the polypeptide B are shown in SEQ ID NO:13;

2) The specific sequences of the polypeptide A are shown in SEQ ID NO:11, specific sequences of the polypeptide B are shown in SEQ ID NO:13;

3) The specific sequences of the polypeptide A are shown in SEQ ID NO:12, specific sequences of the polypeptide B are shown in SEQ ID NO:13;

4) The specific sequences of the polypeptide A are shown in SEQ ID NO:10, specific sequences of the polypeptide B are shown in SEQ ID NO:14;

5) The specific sequences of the polypeptide A are shown in SEQ ID NO:11, specific sequences of the polypeptide B are shown in SEQ ID NO:14;

6) The specific sequences of the polypeptide A are shown in SEQ ID NO:12, specific sequences of the polypeptide B are shown in SEQ ID NO:14.

Wherein said sgRNA nucleotide sequences of CRISPR/Cas9n targeted cutting system in step 2) include identification of specific DNA sequence segments and skeletal RNA fragments on a chromosome, the nucleotide sequences which identify the specific DNA sequence segments are shown in 1) or 2):

1) The nucleotide sequences are shown in SEQ ID NO:15 or SEQ ID NO:16;

2) The nucleotide sequences of the 1) are replaced by one or a few bases and/or deleted and/or added and have the same function as the nucleotide sequences in the 1).

Wherein said sgRNA nucleotide sequences of CRISPR/Cas9n targeted cutting system in step 2) compose of sgRNA-L and sgRNA-R, the sequences of sgRNA-L and sgRNA-R respectively including identification of specific DNA sequence segments and skeletal RNA fragments on a chromosome;

The nucleotide sequences of sgRNA-L which identify the specific DNA sequence segments on a chromosome are shown in 1) or 2):

1) The nucleotide sequences are shown in SEQ ID NO:17;

2) The nucleotide sequences of the 1) are replaced by one or a few bases and/or deleted and/or added and have the same function as the nucleotide sequences in the 1);

The nucleotide sequences of sgRNA-R which identify the specific DNA sequence segments on a chromosome are shown in 3) or 4):

3) The nucleotide sequences are shown in SEQ ID NO:18;

4) The nucleotide sequences of the 3) are replaced by one or a few bases and/or deleted and/or added and have the same function as the nucleotide sequences in the 1).

Wherein the DNA sequences encoding said polypeptide sequences of TALEN targeted cutting system in step 2) include DNA molecular A and DNA molecular B, the specific sequences are shown in 1), 2), 3), 4), 5) or 6):

1) The specific sequences of DNA molecular A which encode the polypeptide shown in SEQ ID NO:10 are shown in SEQ ID NO:19, and the specific sequences of DNA molecular B which encode the polypeptide shown in SEQ ID NO:13 are shown in SEQ ID NO:22;

2) The specific sequences of DNA molecular A which encode the polypeptide shown in SEQ ID NO:11 are shown in SEQ ID NO:20, and the specific sequences of DNA molecular B which encode the polypeptide shown in SEQ ID NO:13 are shown in SEQ ID NO:22;

3) The specific sequences of DNA molecular A which encode the polypeptide shown in SEQ ID NO:12 are shown in SEQ ID NO:21, and the specific sequences of DNA molecular B which encode the polypeptide shown in SEQ ID NO:13 are shown in SEQ ID NO:22;

4) The specific sequences of DNA molecular A which encode the polypeptide shown in SEQ ID NO:10 are shown in SEQ ID NO:19, and the specific sequences of DNA molecular B which encode the polypeptide shown in SEQ ID NO:14 are shown in SEQ ID NO:23;

5) The specific sequences of DNA molecular A which encode the polypeptide shown in SEQ ID NO:11 are shown in SEQ ID NO:20, and the specific sequences of DNA molecular B which encode the polypeptide shown in SEQ ID NO:14 are shown in SEQ ID NO:23;

6) The specific sequences of DNA molecular A which encode the polypeptide shown in SEQ ID NO:12 are shown in SEQ ID NO:21, and the specific sequences of DNA molecular B which encode the polypeptide shown in SEQ ID NO:14 are shown in SEQ ID NO:23.

Further, the DNA molecules encoding said sgRNA nucleotide sequences of CRISPR/Cas9n targeted cutting system in step 2) are the DNA molecules encoding said SEQ ID NO:15 or the DNA molecules encoding said SEQ ID NO:16, the nucleotide sequences of which are show in 1) or 2):

1) The nucleotide sequences are shown in SEQ ID NO:24;

2) The nucleotide sequences are shown in SEQ ID NO:25.

The DNA molecules encoding said sgRNA of CRISPR/Cas9n targeted cutting system in step 2) compose of the DNA molecules A encoding said sgRNA-L and the DNA molecules B encoding said sgRNA-R;

Wherein the nucleotide sequences of DNA molecules A are shown in SEQ ID NO:26, and the nucleotide sequences of DNA molecules B are shown in SEQ ID NO:27.

Wherein said construction of targeting vector in step 3) include the construction of targeting vector with site-specific cleavage and the targeting vector to insert the gene.

Wherein the steps of construction of targeting vector to insert the gene aimed at site-specific cleavage system are as follows: 1) design of the 5′ terminal homology arm and 3′ terminal homology arm with their gene knocked out and the corresponding universal primers; 2) obtain the targeting vector by leading said homology arms, universal primers, marker gene and/or genes to be inserted into the carrier.

Wherein said 5′ terminal homology arm and 3′ terminal homology arm in the step 1) on construction of targeting vector to insert the gene, wherein the nucleotide sequences of the 5′ terminal homology arm are shown in SEQ ID NO:28, and the nucleotide sequences of corresponding universal primers are shown in SEQ ID NO:29; the nucleotide sequences of the 3′ terminal homology arm are shown in SEQ ID NO:30, and the nucleotide sequences of corresponding universal primers are shown in SEQ ID NO:31.

Wherein the sequences of targeting vector to insert the gene constructed for site-specific cleavage system include above mentioned the sequences of 5′ terminal homology, the universal primers sequences of 5′ terminal homology, the gene sequences to be inserted, the universal primers sequences of 3′ terminal homology, the sequences of 3′ terminal homology.

Wherein the nucleotide sequences of targeting vector to insert the gene constructed for site-specific cleavage system are shown in SEQ ID NO:32.

Wherein the nucleotide sequences of PCR amplified primers used in PCR amplification to identify insertion results in step 4) are shown in SEQ ID NO:33, SEQ ID NO:34, SEQ ID NO:35, SEQ ID NO:36, SEQ ID NO:37, SEQ ID NO:38.

Another object of the present invention is to provide the application of the said method in targeted modification of porcine H11 gene.

Another object of the present invention is to provide the application of the said method in the construction of porcine H11 gene mutation library.

The invention provides a method of site-directed insertion to H11 locus in pigs by using site-directed cutting system to achieve a simple, fast and efficient gene insertion. The invention is dependent on the targeting vector designed by cutting system for porcine H11 site, it can introduce the foreign gene into the H11 locus of pig accurately, in order to solve the problems such as low efficiency of traditional shooting technique, inconvenience design of PCR detection primer, harder to detect, and it is efficient, at the same time, the general detection primers are designed according to this site, to greatly reduce the difficulty of screening detection.

Also known by way of examples, said transfect cells of targeting vector, positive clones are screened by the culture media containing the corresponding drugs with positive screening genes, the positive clones are enriched with high efficiency, cell selection method is simple, do not need a lot of manpower and material resources, the subsequent cellular cryopreservation and identification is greatly facilitated, greatly reduced the cost of gene targeting, at the same time, the foreign gene can be stably expressed in H11, to build a stable platform for transgene.

### BRIEF DESCRIPTION OF THE DRAWINGS

FIG. 1 is the structure schematic of targeting vector of the present invention;

FIG. 2 are the identification results of PCR amplification of recombinant cell DNA constructed by TALEN targeted cutting system;

FIG. 3 are the identification results of PCR amplification of recombinant cell DNA constructed by CRISPR/cas9n targeted cutting system;

FIG. 4 are the results of sequencing detection and analysis of the DNA enzyme cutting vector of the recombinant cells constructed by CRISPR/cas9n targeted cutting system;

FIG. 5 are the identification results of PCR amplification of the cells obtained by site-directed insertion to porcine H11 site of the green fluorescent protein constructed by CRISPR/cas9n targeted cutting system; and

FIG. 6A and FIG. 6B are fluorescence excitation of positive clones; wherein FIG. 6A shows microscopic observation results of cells under visible light, FIG. 6B shows microscopic observation results of cells under UV light.

### DETAILED DESCRIPTION

The following examples are used to further illustrate the invention, but should not be construed as a limitation to the present invention. Under the precondition of without departing from the spirit and the essence of the invention, the modification or the replacement of the invention belongs to the category of the invention.

As mentioned in the background, in the cultivation of improved varieties of pigs, the foreign genes are randomly inserted into the genome of pigs, with trouble for the following analysis, in order to overcome the defects, in a typical embodiment of the invention, a method of site-directed insertion to H11 locus in pigs by using site-directed cutting system is provided, the method firstly constructs a TALEN targeted cutting system, a CRISPR/Cas targeted cutting system and a CRISPR/cas9n targeted cutting system, the three kinds of cutting system constructed by the invention can effectively identify the porcine H11 site, and use the corresponding nuclease to cut the sequence gene of the porcine H11 site.

Then a targeting vector is designed to the porcine H11 site using the said targeted cutting system, the said targeting vector is obtained by introduce the homologous arms connected with knockout gene on the two terminals and corresponding universal primers and the gene to be inserted to the pLHG-4. The recombinant cells can be obtained by transfect the above targeting vector cells into cells, when using the site-directed gene mutation library contrasted by the said method, we only need to insert the interest gene between the homology arms then the site-directed insertion of the genes to be completed.

The targeting vector obtained by using said method contains the universal primers, greatly reducing the difficulty and workload of the screening test. And there are not the promoters starting a positive screening gene expression on the inside of the two homologous arms, and there are also negative screening genes on the outside of the homologous arms. The said targeting vector transfect cells, then the positive clones are screened by the culture medium containing corresponding drugs with positive screening genes, the positive clones are enriched with high efficiency, the method of cell screening is simple and does not need a lot of manpower and material resources, greatly reducing the cost of gene targeting, at the same time, the foreign gene can be stably expressed at the H11 locus, and a stable platform for the transgene is built.

The beneficial effects of the present invention are described combined with specific examples in detail below.

### Example 1: Construction of Site-Directed Cutting System of Three Porcine H11 Sites

One. Construction of TALEN Site-Directed and Targeted Cutting System

1. Construction of Target Sequence

Find the sequences of porcine H11 site in gene library. The present invention first according to the gene sequence of porcine H11 locus, as follows:

5′-TACTGAAATGTGACCTACTTTCTTATGTTCCTGGAAGTTTAGATCAGGGT GGGCAGCTCTGGG-3′

2. Design of the TALEN Site

At present, the TALEN system uses FokI incision enzyme activity to cut the target gene, because the FokI can play the activity by forming a dipolymer, in the actual operation we should select two adjacent (interval 14-18 base) target sequences (generally more than a dozen bases) to construct respectively TAL identification modules.

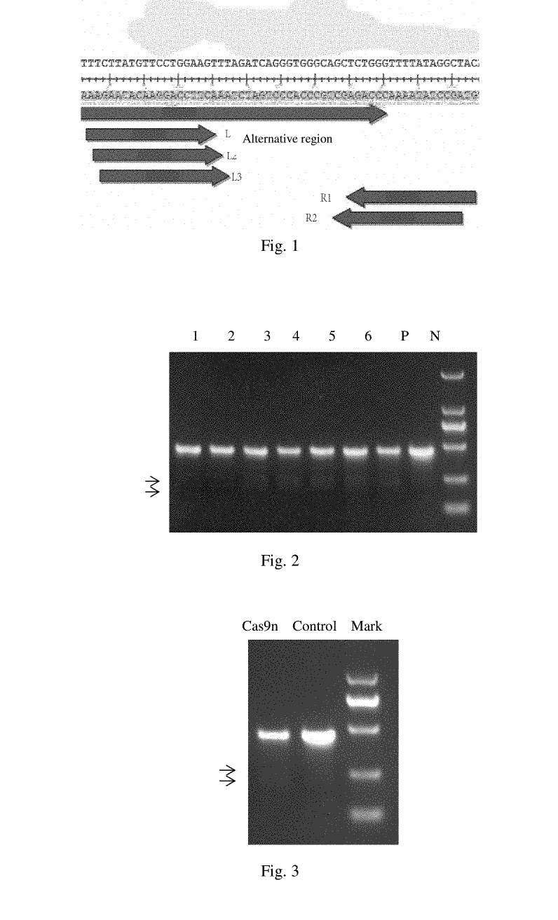

The site of TALEN cutting system is designed according to the target, schematic diagram shown in FIG. 1, the specific sequences are as follows:

L1: 5′-TTCTTATGTTCCTGGAAG-3′ T carrier: L15, the structure of the carrier is: cmv-sp6-NLS-TAL-T-IRES-puro-pA, this carrier is purchased from Shanghai SiDanSai Biotechnology Co., Ltd;

L2: 5′-TCTTATGTTCCTGGAAGT-3′ T carrier: L15, the structure of the carrier is: cmv-sp6-NLS-TAL-T-IRES-puro-pA, this carrier is purchased from Shanghai SiDanSai Biotechnology Co., Ltd;

L3: 5′-CTTATGTTCCTGGAAGTT-3′ T carrier: L15, the structure of the carrier is: cmv-sp6-NLS-TAL-T-IRES-puro-pA, this carrier is purchased from Shanghai SiDanSai Biotechnology Co., Ltd;

R1: 3′-GTAGCCTATAAAACCCAG-5′ A carrier: R10, the structure of the carrier is: cmv-sp6-NLS-TAL-A-pA, this carrier is purchased from Shanghai SiDanSai Biotechnology Co., Ltd;

R2: 3′-AGCCTATAAAACCCAGAG-5′ C carrier: R12, the structure of the carrier is: cmv-sp6-NLS-TAL-C-pA, this carrier is purchased from Shanghai SiDanSai Biotechnology Co., Ltd;

3. TALEN is constructed by using FastTALE™ TALEN rapid construction kit (Cat. No. 1802-030) of Shanghai SiDanSai Biotechnology Co., Ltd, the procedure of construction is:

### (One) Design, Select the Appropriate Module According to the Selected Site, the Design Results are as Follows:

L1:
5′-TTCTTATGTTCCTGGAAG-3′ T carrier: L15
Selected module:
TT1 CT2 TA3 TG4 TT5 CC6 TG7 GA8 AG9
L2:
5′-TCTTATGTTCCTGGAAGT-3′ T carrier: L15
Selected module:
TC1 TT2 AT3 GT4 TC5 CT6 GG7 AA8 GT9
L3:
5′-CTTATGTTCCTGGAAGTT-3′ T carrier: L15
Selected module:
CT1 TA2 TG3 TT4 CC5 TG6 GA7 AG8 TT9
R1:
5′-GTAGCCTATAAAACCCAG-3′ A carrier: R10
Selected module:
GT1 AG2 CC3 TA4 TA5 AA6 AC7 CC8 AG9
R2:
5′-AGCCTATAAAACCCAGAG-3′ C carrier: R12
Selected module:
AG1 CC2 TA3 TA4 AA5 AC6 CC7 AG8 AG9

### (Two) Adding Modules

Add the required modules (a total of 5 tubes) respectively in turn into the 200 ul PCR tube in accordance with the selected modules in the first step.

TABLE 1
module 1

| 1 | 2 | 3 | 4 | 5 | 6 | 7 | 8 | 9 | 10 | 11 | 12 | |
|---|---|---|---|---|---|---|---|---|---|---|---|---|
| AA1 | CA1 | AA2 | CA2 | AA3 | CA3 | AA4 | CA4 | AA5 | CA5 | AA6 | CA6 | A |
| AT1 | CT1 | AT2 | CT2 | AT3 | CT3 | AT4 | CT4 | AT5 | CT5 | AT6 | CT6 | B |
| AC1 | CC1 | AC2 | CC2 | AC3 | CC3 | AC4 | CC4 | AC5 | CC5 | AC6 | CC6 | C |
| AG1 | CG1 | AG2 | CG2 | AG3 | CG3 | AG4 | CG4 | AG5 | CG5 | AG6 | CG6 | D |
| TA1 | GA1 | TA2 | GA2 | TA3 | GA3 | TA4 | GA4 | TA5 | GA5 | TA6 | GA6 | E |
| TT1 | GT1 | TT2 | GT2 | TT3 | GT3 | TT4 | GT4 | TT5 | GT5 | TT6 | GT6 | F |
| TC1 | GC1 | TC2 | GC2 | TC3 | GC3 | TC4 | GC4 | TC5 | GC5 | TC6 | GC6 | G |
| TG1 | GG1 | TG2 | GG2 | TG3 | GG3 | TG4 | GG4 | TG5 | GG5 | TG6 | GG6 | H |

TABLE 2
module 2

| 1 | 2 | 3 | 4 | 5 | 6 | 7 | 8 | 9 | 10 | 11 | 12 | |
|---|---|---|---|---|---|---|---|---|---|---|---|---|
| AA7 | CA7 | AA8 | CA8 | AA9 | CA9 | A1 | T1 | C1 | G1 | | | A |
| AT7 | CT7 | AT8 | CT8 | AT9 | CT9 | A2 | T2 | C2 | G2 | | | B |
| AC7 | CC7 | AC8 | CC8 | AC9 | CC9 | A3 | T3 | C3 | G3 | | | C |
| AG7 | CG7 | AG8 | CG8 | AG9 | CG9 | A4 | T4 | C4 | G4 | | | D |
| TA7 | GA7 | TA8 | GA8 | TA9 | GA9 | A5 | T5 | C5 | G5 | | | E |
| TT7 | GT7 | TT8 | GT8 | TT9 | GT9 | A6 | T6 | C6 | G6 | | | F |
| TC7 | GC7 | TC8 | GC8 | TC9 | GC9 | A7 | T7 | C7 | G7 | | | G |
| TG7 | GG7 | TG8 | GG8 | TG9 | GG9 | | | | | | | H |

### (Three) Adding Sample

Add other solutions respectively into the reagent kit in accordance with the following system, the system is as follows:

TABLE 3
reaction system

| System | | |
|---|---|---|
| Module | 1.5 μL × 9 | |
| Solution1 | 1 | μL |
| Solution2 | 1 | μL |
| Solution3 | 2 | μL |
| Carrier | 1.5 | μL |
| ddH2O | 1 | μL |
| Total volume | 20 | μL |

### (Four) Connect

1) The above mixture is respectively placed on the PCR instrument to complete the connection, the reaction procedure is as follows:

| | | | |
|---|---|---|---|
| 37° C. | 5 min | | |
| | | {close oversize brace} | 15 cycle |
| 16° C. | 10 min | | |

| | |
|---|---|
| 80° C. | 10 min |
| 12° C. | 2 min |

2) Take out the reaction solution in the previous step, respectively add 1 μL solution 4, 0.5 μL solution 5 (total volume 21.5 μL), then incubation for 60 minutes at 37° C.

### (Five) Transform

1) Take out the competence in the kit, and put it on the ice for 10 min to melt.

2) Take 10 μL of the final connection product in step 4 to join in, mixing them.

3) Lay them on the ice for 20 min

4) Heat Shock at 42° C. for 60 s.

5) Ice-bath for 3 min.

6) Add 500 μL SOC, recovery on the shaking table at 37° C. for 30 min.

7) 4000 rpm, centrifugal for 5 min, pour the most supernatant (leave about 150 u L).

8) Resuspend the cells, uniformly coat them on the LB plates resisting kna.

9) Culture at 37° C. for 16 h.

### (Six) Select the Clones

10 clones are selected on the culture plate, cultured in the shaking table at 37° C. overnight (more than 16 h). The primer 305 (5′-CTCCCCTTCAGCTGGACAC-3′) and 306 (5′-AGCTGGGCCACGATTGAC-3′) are sent to the company (Beijing TIANYI HUIYUAN Ltd.) for sequencing, select the correct clones to obtain TALEN: TALEN-H11-L1, TALEN-H11-L2, TALEN-H11-L3, TALEN-H11-R1 and TALEN-H11-R2, extract the plasmid to complete the next experiment.

Two. Construction of CRISPR/Cas9 Targeted Cutting System

1. Find the sequences of porcine H11 site in gene library, select the sgRNA target for gene knockout according to PAM sequence, as follows: 5′-TACTGAAATGTGACCTACTTTCTTATGTTCCTGGAAGTTTAGATCAGGGTGG GCAGCTCTGGG-3′,

Location 1 of sgRNA target site (named as H11-sg1): 5′-GTTCCTGGAAGTTTAGATCAGGG-3′, the nucleotide sequences identifying the target site in the corresponding sgRNA sequences are shown in SEQ ID NO:15, the DNA sequences encoding the above sequences are shown in SEQ ID NO:24.

Location 2 of sgRNA target site (named as H11-sg2): 5′-AGATCAGGGTGGGCAGCTCTGGG-3′, the nucleotide sequences identifying the target site in the corresponding sgRNA sequences are shown in SEQ ID NO:16, the DNA sequences encoding the above sequences are shown in SEQ ID NO:25.

2. Construction of the sgRNA Expression Plasmid

Use the cas9/gRNA construction kit (Catalog. No. VK001-01) of ViewSolid Biotech company to complete the construction, the construction process is as follows:

(1) According to the two target sequences mentioned above, the corresponding primer sequences are designed, synthesized by Beijing TIANYI HUIYUAN Ltd., the specific sequences are shown in Table 4:

TABLE 4
Primer sequences of the two sgRNA targets

| Name of the | |
|---|---|
| nucleotide | Sequences (5′-3′) |
| H11-sg1-F | AAACACCGGTTCCTGGAAGTTTAGATCA |
| H11-sg1-R | CTCTAAAACTGATCTAAACTTCCAGGAAC |
| H11-sg2-F | AAACACCGAGATCAGGGTGGGCAGCTCT |
| H11-sg2-R | CTCTAAAACAGAGCTGCCCACCCTGATCT |

(2) Formation of Oligonucleotide Dipolymer (Oligoduplex)

The synthetic oligo is diluted to 10 μM, mixed in the following proportions

| | |
|---|---|
| H11-sg1-F | 1 μL |
| H11-sg1-R | 1 μL |
| Solution1 | 5 μL |
| H2O | 3 μL |
| Final system | 10 μL |

After mixing respectively, processing in accordance with the following program: 95° C. 3 min; the sample tube is placed in the 95° C. water to cool the above mixture from 95° C. to 25° C.; and then to deal with 5 min at 16° C., finally get the oligonucleotide dipolymer-1.

| | |
|---|---|
| H11-sg2-F | 1 μL |
| H11-sg2-R | 1 μL |
| Solution1 | 5 μL |
| H2O | 3 μL |
| Final system | 10 μL |

After mixing respectively, processing in accordance with the following program: 95° C. 3 min; the sample tube is placed in the 95° C. water to cool the above mixture from 95° C. to 25° C.; and then to deal with 5 min at 16° C., finally get the oligonucleotide dipolymer-2.

(3) The Oligonucleotide Dipolymers are Inserted into the Carrier Respectively

Reaction in the following reaction system:

| | |
|---|---|
| Cas9/gRNA Vector | 1 μL |
| oligoduplex-1 | 2 μL |
| H2O | 7 μL |
| Final system | 10 μL |

After full mixing, standing at room temperature (25° C.). for 5 min, get the carrier Cas9/gRNA-H11-sg1.

| | |
|---|---|
| Cas9/gRNA Vector | 1 μL |
| oligoduplex-2 | 2 μL |
| H2O | 7 μL |
| Final system | 10 μL |

After full mixing, standing at room temperature (25° C.). for 5 min, get the carrier Cas9/gRNA-H11-sg2.

(4) Transform

The final products (carrier Cas9/gRNA-H11-sg1, Cas9/gRNA-H11-sg2) of the step (3) are respectively added into the 50 μL DH5a competent cells which had just thawed, mixing gently, ice bath for 30 min, then heat shock at 42° C. for 90 s, standing on the ice for 2 min, apply directly on the ampicillin resistance plate.

(5) Test and Verify

Pick five white colonies to shake bacteria, and extract the DNA of plasmid for sequencing. The primer for sequencing is 5′-TGAGCGTCGATTTTTGTGATGCTCGTCAG-3′, the sequencing results of Cas9/gRNA-H11-sg2 and Cas9/gRNA-H11-sg1 were obtained, the sequencing results are shown in SEQ ID NO:39 and SEQ ID NO:40. The results indicate that the DNA sequence encoding sgRNA (the sequences of target site 1 and target site 2) can be successfully inserted into the Cas9/gRNA vector backbone by the above operation.

Three. Construction of CRISPR/Cas9n Targeted Cutting System

1. Design the Target

According to the H11 locus of the mouse, find the Eif4 and Drg genes (the site of the mouse is located in the middle of the two genes) of the pig, bring up the middle area in NCBI to find out the H11 site of pig, select the sgRNA target for knocking out the genes according to the PAM sequence (PAM sequence is NGG), as follows:

5′-TACTGAAATGTGACCTACTTTCTTATGTTCCTGGAAGTTTAGATCAG
GGTGGGCAGCTCTGGG-3′

Design the sgRNA target for knocking out the genes: location 1 of SgRNA-L target site (named H11-sgL2): 5′-AGATCAGGGTGGGCAGCTCTGGG-3′, the nucleotide sequences identifying the target in the corresponding sgRNA-L sequence are shown in SEQ ID NO:17; the DNA sequence encoding the above sequences are shown in SEQ ID NO:26.

Location 2 of sgRNA-R target site (named as H11-sgR1): 5′-TTCCAGGAACATAAGAAAGTAGG-3′, the nucleotide sequences identifying the target site in the corresponding sgRNA sequences are shown in SEQ ID NO:18, the DNA sequences encoding the above sequences are shown in SEQ ID NO:27. The two target sequences was “arrangement of head to head”, they are 4 bp apart from each other, that is 4 bp interval.

2. Construction of sgRNA Expression Plasmids

First design the primer sequences according to the target sequence, then send them to Beijing TIANYI HUIYUAN Ltd. to synthetise single-stranded oligonucleotides, specific sequences are as follows:

(1) H11-sgL2:
H11-sgL2-F:
5′-CACCGAGATCAGGGTGGGCAGCTCT-3′
H11-sgL2-R:
5′-AAACAGAGCTGCCCACCCTGATCTC-3′
(2) H11-sgR1:
H11-sgR1-F:
5′-CACCGTTCCAGGAACATAAGAAAGT-3′
H11-sgR1-R:
5′-AAACACTTTCTTATGTTCCTGGAAC-3′

Wherein H11-sgL2-F and H11-sgL2-R were annealed to obtain a double stranded DNA fragment H11-sgL2 with a viscous end, the pX335 (addgene, Plasmid 42335) vector (its nucleotide sequence is as shown in SEQ ID NO:41) is digested by Bbs I enzyme to recover fragment, H11-sgL2 is connected to the fragment to obtain pX335-sgRNA-H11-L vector; H11-sgR1-F and H11-sgR1-R were annealed to obtain a Double stranded DNA fragment H11-gR1 with a viscous end, the pX335 vector is digested by Bbs I enzyme to recover fragment, H11-gR1 is connected to the fragment to obtain pX335-sgRNA-H11-R vector. The two plasmids were sent to Beijing TIANYI HUIYUAN Ltd. to carry out sequencing and verification, the sequence of sequencing primers bbsR is: 5 ‘-GACTATCATATGCTTACCGT-3’, the results of sequencing are respectively show in SEQ ID NO:42 and SEQ ID NO:43. The results show that the sgRNA encoding sequence of the sgRNA target site 1 and the target site 2 of can be inserted into the pX335 vector backbone through the above operation.

### Example 2: Verify the Efficiency of Three Methods for Site-Directed Cutting System of Porcine H11 Sites

1. Separate the Porcine Fetal Fibroblast Cells

PEF cells are isolated from the aborted porcine fetus (methods of separation in reference: Li Hong, Wei Hongjiang, Xu Chengsheng, Wangxia, Qing Yubo, Zeng Yangzhi; Establishment of the fetal fibroblast cell lines of Banna Mini-Pig Inbred and their biological characteristics; Journal of Hunan Agricultural University (natural science ed); Vol. 36, issue 6; in December 2010; 678-682).

2. Eukaryotic Transfection

The recombinant plasmids TALEN-H11-L1 and TALEN-H11-R1, TALEN-H11-L2 and TALEN-H11-R1, TALEN-H11-L3 and TALEN-H11-R1, TALEN-H11-L1 and TALEN-H11-R2, TALEN-H11-L2 and TALEN-H11-R2, TALEN-H11-L3 and TALEN-H11-R2 in example 1, are cotransfected into PEF cells by electroporation in 2.5 μg respectively, to obtain five kinds of recombinant cells. The recombinant plasmids Cas9/gRNA-H11-sg1 and Cas9/gRNA-H11-sg2 obtained in example 1 (Two) are cotransfected into PEF cells by electroporation in 4 μg respectively, to obtain the recombinant cells. The recombinant plasmids pX335-sgRNA-H11-L and pX335-sgRNA-H11-R obtained in example 1 (Three) are cotransfected into PEF cells by electroporation in 2 μg respectively, to obtain a kind of recombinant cell. The specific steps of transfection are: the nuclear transfer instrument (Amaxa, types: AAD-1001S) and a set of transfection kit of mammalian fibroblast cells (Amaxa, No.: VPI-1002) are used to transfect. First use 0.1% trypsin (Gibco, No.: 610-5300AG) to digest adherent cells, use the fetal bovine serum (Gibco, No.: 16000-044) to terminate the digestion, use the phosphate buffer (Gibco, No.: 10010-023) to wash the cells two times, add the transfection reagents, use the procedure T-016 to transfect cells.

3. Extraction of DNA

Eight kinds of recombinant cells could be obtained by step 2, wherein five kinds of recombinant cells obtained in TALEN targeted and site-directed cutting system, two kinds of recombinant cells obtained in CRISPR/Cas9 targeted and site-directed cutting system, a kind of recombinant cell obtained in CRISPR/Cas9n targeted and site-directed cutting system, The above eight kinds of recombinant cells are cultured for 48 hours at 37° C., then collect the cells. The specific steps are: First use 0.1% trypsin (Gibco, No.: 610-5300AG) to digest adherent cells, use the fetal bovine serum (Gibco, No.: 16000-044) to terminate the digestion, use the phosphate buffer (Gibco, No.: 10010-023) to wash the cells two times, add 200 microliters of cell lysate GA (component of DNA extraction kit DP304 in TIANGEN company). Respectively extract the genomic DNA of the above eight kinds of recombinant cells reference the steps of kit manual.

4. Validation of PCR Enzyme Digestion Efficiency

(1) Using the primer H11-F (5′-GCGAGAATTCTAAACTGGAG-3′) and the primer H11-R (5′-GATCTGAGGTGACAGTCTCAA-3′) the PCR amplification is carried out by using five kinds of recombinant cells DNA as template, which are obtained from the TALEN target cutting system in step 3, recovered 387 bp fragment; using the primer H11-F (5′-GCGAGAATTCTAAACTGGAG-3′) and the primer H11-R (5′-GATCTGAGGTGACAGTCTCAA-3′) the PCR amplification was carried out by using two kinds of recombinant cells DNA as template, which were collected from the CRISPR/Cas9 target cutting system in step 3, recovered PCR amplification products of about 370 bp; using the primer H11-F: 5′-GCGAGAATTCTAAACTGGAG-3′ and the primer H11-R: 5′-GATCTGAGGTGACAGTCTCAA-3′ to compose the primer pair, the PCR amplification is carried out by using genomic DNA of recombinant cells as template, which are collected from the CRISPR/Cas9 target cutting system, recovered 387 bp fragment.

The PCR results of recombinant cells of said TALEN target cutting system and CRISPR/Cas9 target cutting system are identified with enzyme cutting by using T7 endonuclease I (T7 endonuclease I, T7E1) (NO: #E001L) of VIewSolid Biotech. Specific steps are:

(2) The PCR products of mutant DNA and wild type DNA are mixed with the following system, and the heat denaturation and annealing treatment are carried out (95° C. 5 min, naturally cooled to room temperature).

TABLE 5
PCR amplification reaction system

| Number | 1 | 2 |
|---|---|---|
| PCR products in the | 5 ul | 0 |
| experimental group | | |
| PCR products in the | 0 | 5 ul |
| control group | | |
| Buffer2 (NEB) | 1.1 ul | 1.1 ul |
| ddH2O | 4.4 ul | 4.4 ul |

| | |
|---|---|
| Total | 10.5 ul |

(3) The 0.5 ul T7E1 enzyme is added to the above reaction system, after reaction at 37° C. for 30 min, enzyme digestion results are detected by 2% agarose gel electrophoresis, the electrophoretogram of the recombinant cells enzyme digestion results of the TALEN target cutting system is shown in FIG. 2, the electrophoretogram of the recombinant cells enzyme digestion results of the CRISPR/Cas9n target cutting system is shown in FIG. 3. Wherein, the Lane 1 in FIG. 2 is TALEN-H11-L1 and TALEN-H11-R1, the Lane 2 is TALEN-H11-L2 and TALEN-H11-R1, the Lane 3 is TALEN-H11-L3 and TALEN-H11-R1, the Lane 4 is TALEN-H11-L1 and TALEN-H11-R2, the Lane 5 is TALEN-H11-L2 and TALEN-H11-R2, the Lane 6 is TALEN-H11-L3 and TALEN-H11-R2, the Lane P is positive transfection Cas9n, the Lane N is control cell. If the TALEN is effective, the target will be cutted out of the 160 bp+230 bp band, target 2 will be cutted out of the 170 bp+220 bp band, the restriction fragment after cutting can be seen from the above figure, and the bands of 3, 4, 5, 6 combination are brighter, the cutting efficiency is higher than 1, 2 groups. Figure of T7EI enzyme digestion: the Lane 1 is TALEN-H11-L1 and TALEN-H11-R1, the Lane 2 is TALEN-H11-L2 and TALEN-H11-R1, the Lane 3 is TALEN-H11-L3 and TALEN-H11-R1, the Lane 4 is TALEN-H11-L1 and TALEN-H11-R2, the Lane 5 is TALEN-H11-L2 and TALEN-H11-R2, the Lane 6 is TALEN-H11-L3 and TALEN-H11-R2, the Lane P is positive transfection Cas9n (introduced in another patent), the Lane N is control cell. If the TALEN is effective, the target will be cutted out of the 160 bp+230 bp band, target 2 will be cutted out of the 170 bp+220 bp band, the restriction fragment after cutting can be seen from the above figure, and the bands of 3, 4, 5, 6 combination are brighter, the efficiency is estimated at about 2%-3%.

From the results of FIG. 3, if the sgRNA is effective, the target position 1 will cutted out the 160 bp+230 bp band, the target position 2 will cutted out the 170 bp+220 bp band, the fuzzy restriction fragment can be seen from the FIG. 3, so the pair of gRNA have certain activity. The specificity of the pair of sgRNA in the cleavage of H11 target site is very strong, which can effectively reduce the miss phenomenon existing in the CRISPR/Cas9 system, greatly increase the efficiency of the fixed point insertion of exogenous gene, and then reduce the impact of the mutation on the non target site of genome caused by nonspecific cleavage.

The identification procedures of cutting results of recombinant cells of CRISPR/Cas9 targeted cutting system are as follows: the PCR amplification product is connected with PMD-18T vector (Takara, No.: D101A), to obtain the connected products, the details of the operation procedures see the description of kit.

The obtained products are transformed into Escherichia coli. DH5a competent cells, and then coated on the LB solid medium plate containing 500 mg/ml ampicillin to culture, 40 clones are randomly selected from two groups respectively and sequenced, proportion of mutant clones in the total number of clones is calculated, so the efficiency of the recombinant plasmid Cas9/gRNA-H11-sg1 and Cas9/gRNA-H11-sg2 plasmid is calculated.

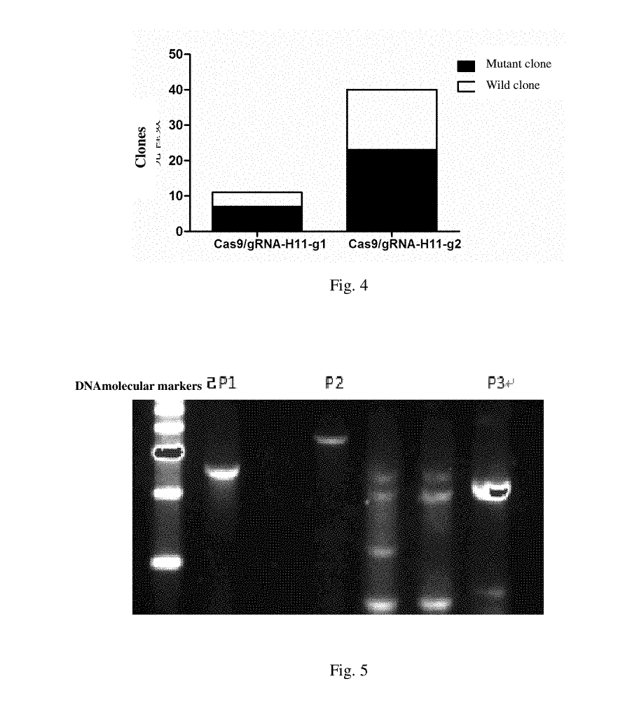

Experimental results are shown in FIG. 4, the results show that: the efficiency of Cas9/gRNA-H11-sg1 is 63% (7 mutants occurred in 11 clones), the efficiency of the Cas9/gRNA-H11-sg2 plasmid is 58% (23 mutants occurred in 40 clones). The results show that the sgRNA could identify the porcine H11 sites efficiently, and carry out fixed point cutting on this site efficiently with the aid of Cas9 enzyme. We can see from the mutation rate of the H11 site of the genomic DNA, for Cas9/gRNA-H11-sg1, its efficiency is 63%, it shows that there are H11 sites of 63 chromosomes in the H11 sites of the 100 chromosomes of the genome identified by the sgRNA, and cutted. In the same way, the efficiency of Cas9/gRNA-H11-sg2 is also very high. It has laid a solid foundation for high efficiency and fixed-point integration experiment to the porcine H11 site.

### Example 3: Method of Fixed-Point Insertion of Green Fluorescent Protein Gene

Method of fixed-point insertion of green fluorescent protein gene to the porcine H11 site with the aid of the CRISPR/Cas9 targeted cutting system constructed by the said target site 1 in the Example 1(Two), comprises the following steps:

1. Construction of Targeting Vector

(1) Synthetic Fragment

According to the DNA sequence of porcine H11 site, design the 3′-terminal homology arm (shown as SEQ ID NO:30), corresponding universal primer (shown as SEQ ID NO:31) and plus the restriction site respectively on two ends: MluI (ACGCGT) and FseI (GGCCGGCC) to join, synthetic fragments are as follows:

5′-ACGCGTttcccgaggctGagttagttgGtccagccagtgattgagt
tgcgtgcggagggcttcttatcttagTTTTATAGGCTACACTGTTAACA
CTCAGGCTGTTTTCTACCGTTTAGTCAAAATATAGTCACCTTGCCTGCT
TCACCTGTCCATCAGAGAATGGCCTCATTAATTGACTCTCTAGTATGAA
GTCAAAGTAGCTTTGGTGGCCCTAAATGGACAAGTATCAAGAGACTGGG
TGAATTGAGGAGCTTGAGACTGTCACCTCAGATCGAAAAGACTGAAAAA
TCACCTCAGATCAAAAAGACTGAAAAATCTTCAGTCTGGAAAGGGGACT
CAAAACCATAATTAGAGTATTCTGGTAGAATCCTTTTCTCCACTGTTAT
TCATACAGTTAAGGTGAATAACTAAAAGTAATTGTGAGCTGAGGAGTAA
GATACAACACACAAGGAATCAGTTAACAGAGTCTCGAGTGAAATTATAA
ATGGAAAGAATTATGACTTGAATCATAACTCTGAGGCCCCATTTTCCCT
AACAACTTTTGTCCCAATAAACGTGGGTATTTGTTTGGGAGAAACTATC
ATATACATGATTACCCAGTAAACAGACTGTTTACTAAGTGGGTTTAATT
TTAGAAATTGCGCGCTGCAATCTGGTATTAACCATACAACTACCTACCT
ATAGGGTCAGCCCAGCCTGAACTATCCCATTGGGGTCTTTATTAAGGCT
CAAGAAACGGCCATAGCTTCTTCCTTTAAAATGAGTGTTTATTTCTATG
AGCTTTAAAGAAAAAAACAGATAATTTCCCTCAACCTACTGAAGAGGAA
GGGATTCAGGAAGAAATAAACACAACAATGCCATTCACTTCAGGCCGGC
C-3′

(2) The DNA fragments obtained in the previous step are cutted into the vector pLHG-4 by MluI (ACGCGT) and FseI (GGCCGGCC) (recovering the fragment of the about 9 KB size, pLHG-4 sequences are shown in SEQ ID NO:44) (PLHG-4 construction steps see Dr. Li Hegang's thesis), the vector named pLHG-H11-AR, the sequences are as follows:

5′-CTATAGTGAGTCGTATTACGCGCGCTCACTGGCCGTCGTTTTACAA
CGTCGTGACTGGGAAAACCCTGGCGTTACCCAACTTAATCGCCTTGCAG
CACATCCCCCTTTCGCCAGCTGGCGTAATAGCGAAGAGGCCCGCACCGA
TCGCCCTTCCCAACAGTTGCGCAGCCTGAATGGCGAATGGGACGCGCCC
TGTAGCGGCGCATTAAGCGCGGCGGGTGTGGTGGTTACGCGCAGCGTGA
CCGCTACACTTGCCAGCGCCCTAGCGCCCGCTCCTTTCGCTTTCTTCCC
TTCCTTTCTCGCCACGTTCGCCGGCTTTCCCCGTCAAGCTCTAAATCGG
GGGCTCCCTTTAGGGTTCCGATTTAGTGCTTTACGGCACCTCGACCCCA
AAAAACTTGATTAGGGTGATGGTTCACGTAGTGGGCCATCGCCCTGATA
GACGGTTTTTCGCCCTTTGACGTTGGAGTCCACGTTCTTTAATAGTGGA
CTCTTGTTCCAAACTGGAACAACACTCAACCCTATCTCGGTCTATTCTT
TTGATTTATAAGGGATTTTGCCGATTTCGGCCTATTGGTTAAAAAATGA
GCTGATTTAACAAAAATTTAACGCGAATTTTAACAAAATATTAACGCTT
ACAATTTAGGTGGCACTTTTCGGGGAAATGTGCGCGGAACCCCTATTTG
TTTATTTTTCTAAATACATTCAAATATGTATCCGCTCATGAGACAATAA
CCCTGATAAATGCTTCAATAATATTGAAAAAGGAAGAGTATGAGTATTC
AACATTTCCGTGTCGCCCTTATTCCCTTTTTTGCGGCATTTTGCCTTCC
TGTTTTTGCTCACCCAGAAACGCTGGTGAAAGTAAAAGATGCTGAAGAT
CAGTTGGGTGCACGAGTGGGTTACATCGAACTGGATCTCAACAGCGGTA
AGATCCTTGAGAGTTTTCGCCCCGAAGAACGTTTTCCAATGATGAGCAC
TTTTAAAGTTCTGCTATGTGGCGCGGTATTATCCCGTATTGACGCCGGG
CAAGAGCAACTCGGTCGCCGCATACACTATTCTCAGAATGACTTGGTTG
AGTACTCACCAGTCACAGAAAAGCATCTTACGGATGGCATGACAGTAAG
AGAATTATGCAGTGCTGCCATAACCATGAGTGATAACACTGCGGCCAAC
TTACTTCTGACAACGATCGGAGGACCGAAGGAGCTAACCGCTTTTTTGC
ACAACATGGGGGATCATGTAACTCGCCTTGATCGTTGGGAACCGGAGCT
GAATGAAGCCATACCAAACGACGAGCGTGACACCACGATGCCTGTAGCA
ATGGCAACAACGTTGCGCAAACTATTAACTGGCGAACTACTTACTCTAG
CTTCCCGGCAACAATTAATAGACTGGATGGAGGCGGATAAAGTTGCAGG
ACCACTTCTGCGCTCGGCCCTTCCGGCTGGCTGGTTTATTGCTGATAAA
TCTGGAGCCGGTGAGCGTGGGTCTCGCGGTATCATTGCAGCACTGGGGC
CAGATGGTAAGCCCTCCCGTATCGTAGTTATCTACACGACGGGGAGTCA
GGCAACTATGGATGAACGAAATAGACAGATCGCTGAGATAGGTGCCTCA
CTGATTAAGCATTGGTAACTGTCAGACCAAGTTTACTCATATATACTTT
AGATTGATTTAAAACTTCATTTTTAATTTAAAAGGATCTAGGTGAAGAT
CCTTTTTGATAATCTCATGACCAAAATCCCTTAACGTGAGTTTTCGTTC
CACTGAGCGTCAGACCCCGTAGAAAAGATCAAAGGATCTTCTTGAGATC
CTTTTTTTCTGCGCGTAATCTGCTGCTTGCAAACAAAAAAACCACCGCT
ACCAGCGGTGGTTTGTTTGCCGGATCAAGAGCTACCAACTCTTTTTCCG
AAGGTAACTGGCTTCAGCAGAGCGCAGATACCAAATACTGTCCTTCTAG
TGTAGCCGTAGTTAGGCCACCACTTCAAGAACTCTGTAGCACCGCCTAC
ATACCTCGCTCTGCTAATCCTGTTACCAGTGGCTGCTGCCAGTGGCGAT
AAGTCGTGTCTTACCGGGTTGGACTCAAGACGATAGTTACCGGATAAGG
CGCAGCGGTCGGGCTGAACGGGGGGTTCGTGCACACAGCCCAGCTTGGA
GCGAACGACCTACACCGAACTGAGATACCTACAGCGTGAGCTATGAGAA
AGCGCCACGCTTCCCGAAGGGAGAAAGGCGGACAGGTATCCGGTAAGCG
GCAGGGTCGGAACAGGAGAGCGCACGAGGGAGCTTCCAGGGGGAAACGC
CTGGTATCTTTATAGTCCTGTCGGGTTTCGCCACCTCTGACTTGAGCGT
CGATTTTTGTGATGCTCGTCAGGGGGGCGGAGCCTATGGAAAAACGCCA
GCAACGCGGCCTTTTTACGGTTCCTGGCCTTTTGCTGGCCTTTTGCTCA
CATGTTCTTTCCTGCGTTATCCCCTGATTCTGTGGATAACCGTATTACC
GCCTTTGAGTGAGCTGATACCGCTCGCCGCAGCCGAACGACCGAGCGCA
GCGAGTCAGTGAGCGAGGAAGCGGAAGAGCGCCCAATACGCAAACCGCC
TCTCCCCGCGCGTTGGCCGATTCATTAATCAGCTGGCACGACAGGTTTC
CCGACTGGAAAGCGGGCAGTGAGCGCAACGCAATTAATGTGAGTTAGCT
CACTCATTAGGCACCCCAGGCTTTACACTTTATGCTTCCGGCTCGTATG
TTGTGTGGAATTGTGAGCGGATAACAATTTCACACAGGAAACAGCTATG
ACCATGATTACGCCAAGCTCGAAATTAACCCTCACTAAAGGGAACAAAA
GCTGGAGCTACTTAAGGGCGCGCCATGAGATGAACTGCTCTGGGATGCC
TAGGTAAATTTCTCTGCATTTCAGTTTCTTTTTAGGAAAGTCAGAACTG
TTCCTTGCAAGATGAGTTCTGAGAACAGAATGTGTTGCAGAAAGTACTG
GAGTCTTTCTAAAAATTTATCCTATGATATTTCCAAGAGACATGGTCAC
CCTTAAGCAAAGTTATACAAGTATTCATGGTCAATTAATACCATTTGGG
GGGGTGTCTTTTTTCTAGGGCTGCACCCATAGCATAAGGAGGTTCCCAG
GAGGTGTGGCCGTCAGCTTATGCCACAACCACAGAAACACCAGATCCAA
GCGGCATCTGTGACCTATACCACAGCTCATAGCAACGCCAGATCCTTAG
CCCCCTTGATTAAAGCCAGGGATCAAACCTGCCTCCTCAAGGATGCTAG
TCAGACTCGTTTACTCTGAGCCACGACAGGAACTCCAAGTAATACCATT
TTTAATCTGGAAAAAAATCTAAATATCATTAAATCCAACCTTGTTATTA
TAAAAGAAGGTACCCCATAGCAAAGGTAGCTAATTCATTCAACTAATGT
GCAGCTCATTAAGGGTGGAGCTGGGAAGTGAGATCTCCTACTTAGCGTC
ACATGCCACCTTGCCTAATAATGATGTATTTGTCTATCAAATGCCTACA
AAGACATACAGAGTCTCTCCCTGGACAGTTTTCATTTTATTATGTGATC
GTTACTACCCCAAAGATTTCTTTCTTGATTTTATTTTGTCCCTCATATT
CTGTCTGTCATCCCTACATTCAGATATCAGAGGTGGGGGTATTGGGGAG
GGGGAGATGAGGAGAGGAAAAGGATTGGTTGGTGCATGGCCAGTCAAGT
TGAAGATGACTGCAACAATCACGAGAAATCTCTGCAAAACTATAAAAGC
TTCCTGGGGTGCCTTCTGAAAAAGTCTGATCCAAGTTGCTTTATTAGGG
CCTGGACCATTTCTAGAAGTAGATGAATGCATTCCTTTCATTGGCTAGG
AGGTGGGGATGGGGCAGAGAGCATACTTCTGTTTCTGCAGCTGAGACCT
GGACATGGTGAACCTGGAGTAGCTACCCATATGGCATGGACAGGTCCAA
CTGCTGCCCCCTCCTTTGTCCCCCAAGAAGCCAGCAGGGGCAGGATGAA
GGCCACCTTGGGGCTGCCCTGAGCCTCCTGCAGTATGCCTGGCAACTAC
TTTCTTAGCCATCTTTAAGGCCCAATCTTGGGTAAAATACTACTCAACC
CATTCTTTAGCCACCTTCTCCAAATGCTTCTAGAAAGCGGCCCCCACAA
GTAGGTTCTCTGCAGCAGCACAGTGCAAATGGAGGAACACGACCTCAGT
AATTATTTTGTCACTGCAAAGTATCTACAACCTTTGCTATAAAAATTAA
CACCTTGCTTTCCCTGAAAAATAGCCCAGTCATATCCAGCATTTTCCAG
CATCCAGGGCAGAGTGCTTGCTCCTCCCCCAGTCAACAGGACTGTTCAT
ACCGAGGAAATGATTTGAGGGTTCTTTAAGCATTTACGCTGTTAATGCT
AAAGCTTTCACGACTTCTACCTGAGGGGGGCTTGAGGGAGGGGGGAGGT
TTATGTCCCTGCACCGCCAGGAGCCTGGTCTTTGGTAGGAACGCAGAGG
CAGCCGGCGACCTTCCACCCTCAGTGTGTCCTTCCCCAGGAGTTTAGGG
AAGTGAATCCCTAGATCCAGCCAACATTTCCACTCCCATTTTCAAGAGA
TTAAAAAAAAAAAAAAAAAAAAAAAAAAGGAAAGCATCGGCAGGTCAGC
AAACCAGCAGTTCTCCATCCTTGGGATCTTAGCAGCCGACGACCTTAAT
TAAACGCGGTGGCGGCCGCATTACCCTGTTATCCCTAGAATTCGATGCT
GAAGTTCCTATAGTTTCTAGAGTATAGGAACTTCGGTCATAACTTCGTA
TAGCATACATTATACGAAGTTATTCCGGATAAGATACATTGATGAGTTT
GGACAAACCACAACTAGAATGCAGTGAAAAAAATGCTTTATTTGTGAAA
TTTGTGATGCTATTGCTTTATTTGTAACCATTATAAGCTGCAATAAACA
AGTTGGGGTGGGCGAAGAACTCCAGCATGAGATCCCCGCGCTGGAGGAT
CATCCAGCCGGCGTCCCGGAAAACGATTCCGAAGCCCAACCTTTCATAG
AAGGCGGCGGTGGAATCGAAATCTCGTGATGGCAGGTTGGGCGTCGCTT
GGTCGGTCATTTCGAACCCCAGAGTCCCGCTCAGAAGAACTCGTCAAGA
AGGCGATAGAAGGCGATGCGCTGCGAATCGGGAGCGGCGATACCGTAAA
GCACGAGGAAGCGGTCAGCCCATTCGCCGCCAAGCTCTTCAGCAATATC
ACGGGTAGCCAACGCTATGTCCTGATAGCGGTCCGCCACACCCAGCCGG
CCACAGTCGATGAATCCAGAAAAGCGGCCATTTTCCACCATGATATTCG
GCAAGCAGGCATCGCCATGGGTCACGACGAGATCCTCGCCGTCGGGCAT
GCGCGCCTTGAGCCTGGCGAACAGTTCGGCTGGCGCGAGCCCCTGATGC
TCTTCGTCCAGATCATCCTGATCGACAAGACCGGCTTCCATCCGAGTAC
GTGCTCGCTCGATGCGATGTTTCGCTTGGTGGTCGAATGGGCAGGTAGC
CGGATCAAGCGTATGCAGCCGCCGCATTGCATCAGCCATGATGGATACT
TTCTCGGCAGGAGCAAGGTGAGATGACAGGAGATCCTGCCCCGGCACTT
CGCCCAATAGCAGCCAGTCCCTTCCCGCTTCAGTGACAACGTCGAGCAC
AGCTGCGCAAGGAACGCCCGTCGTGGCCAGCCACGATAGCCGCGCTGCC
TCGTCCTGCAGTTCATTCAGGGCACCGGACAGGTCGGTCTTGACAAAAA
GAACCGGGCGCCCCTGCGCTGACAGCCGGAACACGGCGGCATCAGAGCA
GCCGATTGTCTGTTGTGCCCAGTCATAGCCGAATAGCCTCTCCACCCAA
GCGGCCGGAGAACCTGCGTGCAATCCATCTTGTTCAATCATGCGAAACG
ATCCTCATGCTAGCTTATCATCGTGTTTTTCAAAGGAAAACCACGTCCC
CGTGGTTCGGGGGGCCTAGACGTTTTTTTAACCTCGACTAAACACATGT
AAAGCATGTGCACCGAGGCCCCAGATCAGATCCCATACAATGGGGTACC
TTCTGGGCATCCTTCAGCCCCTTGTTGAATACGCTTGAGGAGAGCCATT
TGACTCTTTCCACAACTATCCAACTCACAACGTGGCACTGGGGTTGTGC
CGCCTTTGCAGGTGTATCTTATACACGTGGCTTTTGGCCGCAGAGGCAC
CTGTCGCCAGGTGGGGGGTTCCGCTGCCTGCAAAGGGTCGCTACAGACG
TTGTTTGTCTTCAAGAAGCTTCCAGAGGAACTGCTTCCTTCACGACATT
CAACAGACCTTGCATTCCTTTGGCGAGAGGGGAAAGACCCCTAGGAATG
CTCGTCAAGAAGACAGGGCCAGGTTTCCGGGCCCTCACATTGCCAAAAG
ACGGCAATATGGTGGAAAATAACATATAGACAAACGCACACCGGCCTTA
TTCCAAGCGGCTTCGGCCAGTAACGTTAGGGGGGGGGGGGGAGAGGGGC
GGAATTGGATCCGATATCTTACTTGTACAGCTCGTCCATGCCGAGAGTG
ATCCCGGCGGCGGTCACGAACTCCAGCAGGACCATGTGATCGCGCTTCT
CGTTGGGGTCTTTGCTCAGGGCGGACTGGGTGCTCAGGTAGTGGTTGTC
GGGCAGCAGCACGGGGCCGTCGCCGATGGGGGTGTTCTGCTGGTAGTGG
TCGGCGAGCTGCACGCTGCCGTCCTCGATGTTGTGGCGGATCTTGAAGT
TCACCTTGATGCCGTTCTTCTGCTTGTCGGCCATGATATAGACGTTGTG
GCTGTTGTAGTTGTACTCCAGCTTGTGCCCCAGGATGTTGCCGTCCTCC
TTGAAGTCGATGCCCTTCAGCTCGATGCGGTTCACCAGGGTGTCGCCCT
CGAACTTCACCTCGGCGCGGGTCTTGTAGTTGCCGTCGTCCTTGAAGAA
GATGGTGCGCTCCTGGACGTAGCCTTCGGGCATGGCGGACTTGAAGAAG
TCGTGCTGCTTCATGTGGTCGGGGTAGCGGCTGAAGCACTGCACGCCGT
AGGTCAGGGTGGTCACGAGGGTGGGCCAGGGCACGGGCAGCTTGCCGGT
GGTGCAGATGAACTTCAGGGTCAGCTTGCCGTAGGTGGCATCGCCCTCG
CCCTCGCCGGACACGCTGAACTTGTGGCCGTTTACGTCGCCGTCCAGCT
CGACCAGGATGGGCACCACCCCGGTGAACAGCTCCTCGCCCTTGCTCAC
CATCTTAAGGATCTGACGGTTCACTAAACCAGCTCTGCTTATATAGACC
TCCCACCGTACACGCCTACCGCCCATTTGCGTCAATGGGGCGGAGTTGT
TACGACATTTTGGAAAGTCCCGTTGATTTTGGTGCCAAAACAAACTCCC
ATTGACGTCAATGGGGTGGAGACTTGGAAATCCCCGTGAGTCAAACCGC
TATCCACGCCCATTGATGTACTGCCAAAACCGCATCACCATGGTAATAG
CGATGACTAATACGTAGATGTACTGCCAAGTAGGAAAGTCCCATAAGGT
CATGTACTGGGCATAATGCCAGGCGGGCCATTTACCGTCATTGACGTCA
ATAGGGGGCGTACTTGGCATATGATACACTTGATGTACTGCCAAGTGGG
CAGTTTACCGTAAATACTCCACCCATTGACGTCAATGGAAAGTCCCTAT
TGGCGTTACTATGGGAACATACGTCATTATTGACGTCAATGGGCGGGGG
TCGTTGGGCGGTCAGCCAGGCGGGCCATTTACCGTAAGTTATGTAACGC
GGAACTCCATATATGGGCTATGAACTAATGACCCCGTAATTGAGATCTG
AAGTTCCTATAGTTTCTAGAGTATAGGAACTTCGGTCATAACTTCGTAT
AGCATACATTATACGAAGTTATACGCGTttcccgaggctGagttagttg
GtccagccagtgattgagttgcgtgcggagggcttcttatcttagTTTT
ATAGGCTACACTGTTAACACTCAGGCTGTTTTCTACCGTTTAGTCAAAA
TATAGTCACCTTGCCTGCTTCACCTGTCCATCAGAGAATGGCCTCATTA
ATTGACTCTCTAGTATGAAGTCAAAGTAGCTTTGGTGGCCCTAAATGGA
CAAGTATCAAGAGACTGGGTGAATTGAGGAGCTTGAGACTGTCACCTCA
GATCGAAAAGACTGAAAAATCACCTCAGATCAAAAAGACTGAAAAATCT
TCAGTCTGGAAAGGGGACTCAAAACCATAATTAGAGTATTCTGGTAGAA
TCCTTTTCTCCACTGTTATTCATACAGTTAAGGTGAATAACTAAAAGTA
ATTGTGAGCTGAGGAGTAAGATACAACACACAAGGAATCAGTTAACAGA
GTCTCGAGTGAAATTATAAATGGAAAGAATTATGACTTGAATCATAACT
CTGAGGCCCCATTTTCCCTAACAACTTTTGTCCCAATAAACGTGGGTAT
TTGTTTGGGAGAAACTATCATATACATGATTACCCAGTAAACAGACTGT
TTACTAAGTGGGTTTAATTTTAGAAATTGCGCGCTGCAATCTGGTATTA
ACCATACAACTACCTACCTATAGGGTCAGCCCAGCCTGAACTATCCCAT
TGGGGTCTTTATTAAGGCTCAAGAAACGGCCATAGCTTCTTCCTTTAAA
ATGAGTGTTTATTTCTATGAGCTTTAAAGAAAAAAACAGATAATTTCCC
TCAACCTACTGAAGAGGAAGGGATTCAGGAAGAAATAAACACAACAATG
CCATTCACTTCAGGCCGGCCTCTAGAATGCATGTTTAAACAGGCCGCGG
GAATTCGATTATCGAATTCTACCGGGTAGGGGAGGCGCTTTTCCCAAGG
CAGTCTGGAGCATGCGCTTTAGCAGCCCCGCTGGGCACTTGGCGCTACA
CAAGTGGCCTCTGGCCTCGCACACATTCCACATCCACCGGTAGGCGCCA
ACCGGCTCCGTTCTTTGGTGGCCCCTTCGCGCCACCTTCTACTCCTCCC
CTAGTCAGGAAGTTCCCCCCCGCCCCGCAGCTCGCGTCGTGCAGGACGT
GACAAATGGAAGTAGCACGTCTCACTAGTCTCGTGCAGATGGACAGCAC
CGCTGAGCAATGGAAGCGGGTAGGCCTTTGGGGCAGCGGCCAATAGCAG
CTTTGCTCCTTCGCTTTCTGGCTCAGAGGCTGGGAAGGGGTGGGTCCGG
GGGCGGGCTCAGGGGCGGGCTCAGGGGCGGGGCGGGCGCCCGAAGGTCC
TCCGGAGGCCCGGCATTCTGCACGCTTCAAAAGCGCACGTCTGCCGCGC
TGTTCTCCTCTTCCTCATCTCCGGGCCTTTCGACCTGCAGGTCCTCGCC
ATGGATCCTGATGATGTTGTTGATTCTTCTAAATCTTTTGTGATGGAAA
ACTTTTCTTCGTACCACGGGACTAAACCTGGTTATGTAGATTCCATTCA
AAAAGGTATACAAAAGCCAAAATCTGGTACACAAGGAAATTATGACGAT
GATTGGAAAGGGTTTTATAGTACCGACAATAAATACGACGCTGCGGGAT
ACTCTGTAGATAATGAAAACCCGCTCTCTGGAAAAGCTGGAGGCGTGGT
CAAAGTGACGTATCCAGGACTGACGAAGGTTCTCGCACTAAAAGTGGAT
AATGCCGAAACTATTAAGAAAGAGTTAGGTTTAAGTCTCACTGAACCGT
TGATGGAGCAAGTCGGAACGGAAGAGTTTATCAAAAGGTTCGGTGATGG
TGCTTCGCGTGTAGTGCTCAGCCTTCCCTTCGCTGAGGGGAGTTCTAGC
GTTGAATATATTAATAACTGGGAACAGGCGAAAGCGTTAAGCGTAGAAC
TTGAGATTAATTTTGAAACCCGTGGAAAACGTGGCCAAGATGCGATGTA
TGAGTATATGGCTCAAGCCTGTGCAGGAAATCGTGTCAGGCGATCTCTT
TGTGAAGGAACCTTACTTCTGTGGTGTGACATAATTGGACAAACTACCT
ACAGAGATTTAAAGCTCTAAGGTAAATATAAAATTTTTAAGTGTATAAT
GTGTTAAACTACTGATTCTAATTGTTTGTGTATTTTAGATTCCAACCTA
TGGAACTGATGAATGGGAGCAGTGGTGGAATGCAGATCCTAGAGCTCGC
TGATCAGCCTCGACTGTGCCTTCTAGTTGCCAGCCATCTATTGTTTGCC
CCTCCCCCGTGCCTTCCTTGACCCTGGAAGGTGCCACTCCCACTGTCCT
TTCCTAATAAAATGAGGAAATTGCATCGCATTGTCTGAGTAGGTGTCAT
TCTATTCTGGGGGGTGGGGTGGGGCAGGACAGCAAGGGGGAGGATTGGG
AAGACAATAGCAGGCATGCTGGGGATGCGGTGGGCTCTATGGCTTCTGA
GGCGGAAAGAACCAGCTGGGGCTCGAGGGGGGGCCCGGTACCCAATTCG
CC-3′

(3) Synthetic Fragment

According to the DNA sequence of porcine H11 site, design the 5′-terminal homology arm (shown as SEQ ID NO:28), corresponding universal primer (shown as SEQ ID NO:29) and plus RFP encoding sequence, polyA sequence and plus the restriction site respectively on two ends: Asc I (GGCGCGCC), Pac I (TTAATTAA), synthetic fragments are as follows:

5′-GGCGCGCCCATTGAGCCACGAACAGAACTCCCTCTTACCAACTTAT
TACTACTAACTTCCCAAGTACTGGCTGCTCAGCTGCTTCCTTGGGCATG
GGGGAGGGAGCACTATTTTTTCCTCTCCTGACTTCATCCTCTTCCTTTT
AATTTCCATAAGGTTCCCTGTGGCCCTGTGCTTTTTTATTTTGAGGCCT
TGCACATCCTTCTGGCCCTGATTGCTTCTCAACTCATCTTGTGCCTGCT
GGACTTCCACCGTTGTTTCATGTATCTCGTTAGCTGAGATAGCACTTCC
TCCTGCCCTTACCCTTTATCTGGCTCTTAGCTCCTGAAAACTGCATTAT
TAGCTTCCTCTTTTGCCTCTACTCTTACTCAACCAAAATTGTTTTAAGA
TCTGTGGATCTAGCTTCTGCTGTGCTATTCTTAGGAACACTTTTATTTC
CTCTTAGCTCCATCTCACCAGTTATTGGCTAATGGCTTTGCTTGGTACC
TACATCTGTACATTTCTTTCGTACTAGCTTCTAGACTGAAAAAGGACTG
TTGGTTCAACATGAAAGGGAAGGAGGTAAAAGAGGACACACAGGAAAGA
TGGATTGGGATTCAGGTCTCTGCTGTTGTTACTTGAGATTGCTTTCTAG
ATTCTACTTGTGGAAACAAAAAGCCTTTGCGAGAATTCTAAACTGGAGT
ATTTCTGTAATTGAGGAGTCTTGCTCAGCAAATCCCACTTAGGGGACTA
ATGAAGTACCAGGAAGAGACAGACCATGCTCAATCCACAAAGCCAGGTT
TTACTGAAATGTGACCTACTTTCTTATGCGATCGCCTgccgaaagagta
atgTtggCCgagataggagaagacGatgatatcacgctacgacggaaac
AGTACTATGGCCTCCTCCGAGGACGTCATCAAGGAGTTCATGCGCTTCA
AGGTGCGCATGGAGGGCTCCGTGAACGGCCACGAGTTCGAGATCGAGGG
CGAGGGCGAGGGCCGCCCCTACGAGGGCACCCAGACCGCCAAGCTGAAG
GTGACCAAGGGCGGCCCCCTGCCCTTCGCCTGGGACATCCTGTCCCCTC
AGTTCCAGTACGGCTCCAAGGCCTACGTGAAGCACCCCGCCGACATCCC
CGACTACTTGAAGCTGTCCTTCCCCGAGGGCTTCAAGTGGGAGCGCGTG
ATGAACTTCGAGGACGGCGGCGTGGTGACCGTGACCCAGGACTCCTCCC
TGCAGGACGGCGAGTTCATCTACAAGGTGAAGCTGCGCGGCACCAACTT
CCCCTCCGACGGCCCCGTAATGCAGAAGAAGACCATGGGCTGGGAGGCC
TCCACCGAGCGGATGTACCCCGAGGACGGCGCCCTGAAGGGCGAGATCA
AGATGAGGCTGAAGCTGAAGGACGGCGGCCACTACGACGCCGAGGTCAA
GACCACCTACATGGCCAAGAAGCCCGTGCAGCTGCCCGGCGCCTACAAG
ACCGACATCAAGCTGGACATCACCTCCCACAACGAGGACTACACCATCG
TGGAACAGTACGAGCGCGCCGAGGGCCGCCACTCCACCGGCGCCTAAGA
ATGCAATTGTTGTTGTTAACTTGTTTATTGCAGCTTATAATGGTTACAA
ATAAAGCAATAGCATCACAAATTTCACAAATAAAGCATTTTTTTCACTG
CATTCTAGTTGTGGTTTGTCCAAACTCATCAATGTATCTTA
TTAATTAA-3′

(4) Asc I (GGCGCGCC), Pac I (TTAATTAA) double-enzyme digest vector pLHG-H11-AR (recovering of 8 KB size fragments), connected with the DNA fragment obtained from the last step, to obtain the final carrier pLHG-H11, shown as SEQ ID NO:45.

2. Verification of the Vector Efficiency

(1) Separate the Porcine Fetal Fibroblast Cells

PEF cells are isolated from the aborted porcine fetus, the specific separation method see the literature: Li Hong, Wei Hongjiang, Xu Chengsheng, Wangxia, Qing Yubo, Zeng Yangzhi; Establishment of the fetal fibroblast cell lines of Banna Mini-Pig Inbred and their biological characteristics.

(2) Linearization

The pLHG-H11 are linearized using BclI (NEB, R0160S), using the agarose gel extraction kit (DP209) of TIANGEN BIOTECH (BEIJING) CO., LTD, recycling fragments for the next experiment, specific operation method see the kit instructions.

(3) Eukaryotic Transfection

The recombinant plasmids Cas9/gRNA-H11-sg1 and the linearized pLHG-H11 are cotransfected into PEF cells by electroporation in 2.5 μg respectively, to obtain the recombinant cells. The specific steps of transfection are: transfection is carried out by using nuclear instrument (Amaxa and types: AAD-10015) and a set of mammalian fibroblast cells transfection Kit (Amaxa, No.: VPI-1002). First use 0.1% trypsin (Gibco, No.: 610-5300AG) to digest adherent cells, use the fetal bovine serum (Gibco, No.: 16000-044) to terminate the digestion, use the phosphate buffer (Gibco, No.: 10010-023) to wash the cells two times, add the transfection reagents, use the procedure T-016 to transfect cells.

(4) Cell Selection

After the electrotransformation, the recombinant cells are cultured for 72 hours at 30° C., and then the cells are collected. The cells are diluted, a certain number of cells in each of the 10 cm culture dishes, change the culture medium every 2-3 days. FIG. 2 is the clone of planking for 6 days.

After planking for 10 days, the cells begin to form monoclone, the half of cells in each of the monoclonal cells are collected to use for genome extraction, the rest of the cells continue to be cultured. A total of 132 clones are collected.

5) Cell Positive Identification

PCR amplification is performed using the following general primers, and the ampliconic sequences are:

TABLE 6
The primers using for PCR amplification

| Primer | | |
|---|---|---|
| name | Sequences (5′-3′) | Remarks |
| H11-L-F1 | CTCAGTCCCAGGCTTTACATC | Amplification |
| H11-L-R1 | CCAACATTACTCTTTCGGCAG | of the left arm |
| H11-L-F2 | ACTGGCTTTCTGAGTTAGGG | Amplification |
| H11-L-R2 | GTTTCCGTCGTAGCGTGATA | of the left arm |
| H11-R-F3 | CGGAGGGCTTCTTATCTTAG | Amplification |
| H11-R-R3 | GTGTGGAGCTGTTTAGGGAC | of the right |
| | | arm |

Please add the steps of electrophoresis, the electrophoresis results are shown in FIG. 5, the P1 indicate the amplified fragments by the primer H11-L-F1 and H11-L-R1, the size of 1.2 kb, the P2 indicate the amplified fragments by the primer H11-L-F2 and H11-L-R2, the P3 indicate the amplified fragments by the primer H11-R-F3 and H11-R-R3.

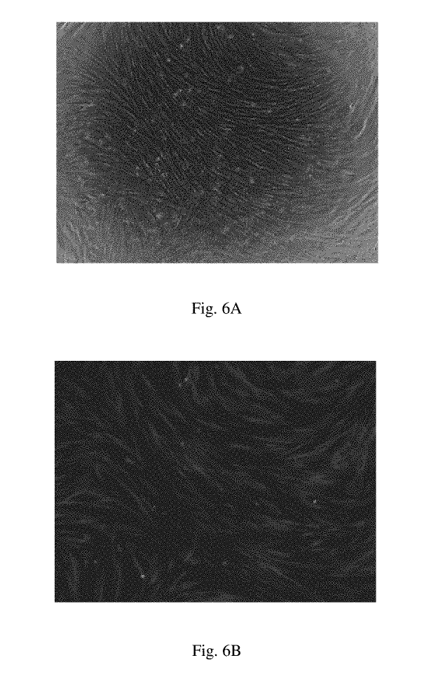

It can be drawn by the PCR identification, 31 positive clones are obtained from 132 clones (all 3 pairs of primer are amplificated), the positive rate is 23%, the screened positive clones are excited under ultraviolet light (blue light), the results are shown in FIG. 6A and FIG. 6B, the screened positive clones can stimulate the green fluorescence from FIGS. 6A and 6B, this shows that the vector can be used well for fixed-point insertion of H11 sites.

1. A method of site-directed insertion to H11 locus in pigs by using site-directed cutting system, which is characterized in that said method includes the following steps:
1) identify the targeted sequence targeted by the targeted cutting system in the targeted genome sequence of pigs;
2) design and construct the targeting sequence of the corresponding cutting system according to the targeted site;
3) construction of targeting vector;
4) transfect cells, identify the efficiency of site-directed insertion by PCR amplification.

2. The method according to claim 1, which is characterized in that, said targeted cutting system in step 1 is a TALEN targeted cutting system or CRISPR/Cas targeted cutting system.

3. The method according to claim 2, which is characterized in that, said nucleotide cleaving enzyme using in CRISPR/Cas target cutting system is csa9 or cas9n.

4. The method according to claim 2, which is characterized in that, said targeted sequence targeted by the targeted cutting system in step 1 is the targeted sequence targeted by the TALEN targeted cutting system, CRISPR/Cas9 targeted cutting system or targeted sequence targeted by CRISPR/Cas9n targeted cutting system.

5. The method according to claim 4, which is characterized in that, said targeted sequences in step 1 are shown in 1), 2) or 3):
1) the targeted sequences targeted by the TALEN targeted cutting system are a pair of sites, having nucleotide sequences shown in SEQ ID NO:1 and SEQ ID NO:4, SEQ ID NO:2 and SEQ ID NO:4, SEQ ID NO:3 and SEQ ID NO:4, SEQ ID NO:1 and SEQ ID NO:5, SEQ ID NO:2 and SEQ ID NO:5, or SEQ ID NO:3 and SEQ ID NO:5;
2) the targeted sequences targeted by CRISPR/Cas9 targeted cutting system are shown in SEQ ID NO:6 or SEQ ID NO:7;
3) the targeted sequences targeted by CRISPR/Cas9n targeted cutting system is a pair of sites, having nucleotide sequences shown in SEQ ID NO:8 and SEQ ID NO:9.

6. The method according to claim 1, which is characterized in that, said targeted sequences in step 2 are polypeptide sequences of a TALEN targeted cutting system, nucleotide sequences of CRISPR/Cas9 targeted cutting system or a pair of nucleotide sequences of CRISPR/Cas9n targeted cutting system.

7. The method according to claim 6, which is characterized in that, said polypeptide sequences of the TALEN targeted cutting system include polypeptide A and polypeptide B, the specific sequences are shown in 1), 2), 3), 4), 5) or 6):
1) the specific sequences of the polypeptide A are shown in SEQ ID NO:10, specific sequences of the polypeptide B are shown in SEQ ID NO:13;
2) the specific sequences of the polypeptide A are shown in SEQ ID NO:11, specific sequences of the polypeptide B are shown in SEQ ID NO:13;
3) the specific sequences of the polypeptide A are shown in SEQ ID NO:12, specific sequences of the polypeptide B are shown in SEQ ID NO:13;
4) the specific sequences of the polypeptide A are shown in SEQ ID NO:10, specific sequences of the polypeptide B are shown in SEQ ID NO:14;
5) the specific sequences of the polypeptide A are shown in SEQ ID NO:11, specific sequences of the polypeptide B are shown in SEQ ID NO:14;
6) the specific sequences of the polypeptide A are shown in SEQ ID NO:12, specific sequences of the polypeptide B are shown in SEQ ID NO:14.

8. The method according to claim 6, which is characterized in that, said sgRNA nucleotide sequences of CRISPR/Cas9n targeted cutting system in step 2) include identification of specific DNA sequence segments and skeletal RNA fragments on a chromosome, the nucleotide sequences which identify the specific DNA sequence segments are shown in 1) or 2):
1) the nucleotide sequences are shown in SEQ ID NO:15 or SEQ ID NO:16;
2) the nucleotide sequences of the 1) are replaced by one or a few bases and/or deleted and/or added and have the same function as the nucleotide sequences in the 1).

9. The method according to claim 6, which is characterized in that, said sgRNA nucleotide sequences of CRISPR/Cas9n targeted cutting system in step 2) compose of sgRNA-L and sgRNA-R, the sequences of sgRNA-L and sgRNA-R respectively including identification of specific DNA sequence segments and skeletal RNA fragments on a chromosome;
the nucleotide sequences of sgRNA-L which identify the specific DNA sequence segments on a chromosome are shown in 1) or 2):
1) the nucleotide sequences are shown in SEQ ID NO:17;
2) the nucleotide sequences of the 1) are replaced by one or a few bases and/or deleted and/or added and have the same function as the nucleotide sequences in the 1);
the nucleotide sequences of sgRNA-R which identify the specific DNA sequence segments on a chromosome are shown in 3) or 4):
3) the nucleotide sequences are shown in SEQ ID NO:18;
4) the nucleotide sequences of the 3) are replaced by one or a few bases and/or deleted and/or added and have the same function as the nucleotide sequences in the 3).

10. The method according to claim 7, which is characterized in that, the DNA sequences encoding said polypeptide sequences of the TALEN targeted cutting system in step 2) include DNA molecular A and DNA molecular B, the specific sequences are shown in 1), 2), 3), 4), 5) or 6):
1) the specific sequences of DNA molecular A which encode the polypeptide shown in SEQ ID NO:10 are shown in SEQ ID NO:19, and the specific sequences of DNA molecular B which encode the polypeptide shown in SEQ ID NO:13 are shown in SEQ ID NO:22;
2) the specific sequences of DNA molecular A which encode the polypeptide shown in SEQ ID NO:11 are shown in SEQ ID NO:20, and the specific sequences of DNA molecular B which encode the polypeptide shown in SEQ ID NO:13 are shown in SEQ ID NO:22;
3) the specific sequences of DNA molecular A which encode the polypeptide shown in SEQ ID NO:12 are shown in SEQ ID NO:21, and the specific sequences of DNA molecular B which encode the polypeptide shown in SEQ ID NO:13 are shown in SEQ ID NO:22;
4) the specific sequences of DNA molecular A which encode the polypeptide shown in SEQ ID NO:10 are shown in SEQ ID NO:19, and the specific sequences of DNA molecular B which encode the polypeptide shown in SEQ ID NO:14 are shown in SEQ ID NO:23;
5) the specific sequences of DNA molecular A which encode the polypeptide shown in SEQ ID NO:11 are shown in SEQ ID NO:20, and the specific sequences of DNA molecular B which encode the polypeptide shown in SEQ ID NO:14 are shown in SEQ ID NO:23;
6) the specific sequences of DNA molecular A which encode the polypeptide shown in SEQ ID NO:12 are shown in SEQ ID NO:21, and the specific sequences of DNA molecular B which encode the polypeptide shown in SEQ ID NO:14 are shown in SEQ ID NO:23.

11. The method according to claim 8, which is characterized in that, the DNA molecules encoding said sgRNA nucleotide sequences of CRISPR/Cas9n targeted cutting system in step 2) are the DNA molecules encoding said SEQ ID NO:15 or the DNA molecules encoding said SEQ ID NO:16, the nucleotide sequences of which are show in 1) or 2):
1) the nucleotide sequences are shown in SEQ ID NO:24;
2) the nucleotide sequences are shown in SEQ ID NO:25.

12. The method according to claim 9, which is characterized in that, the DNA molecules encoding said sgRNA of CRISPR/Cas9n targeted cutting system in step 2) compose of the DNA molecules A encoding said sgRNA-L and the DNA molecules B encoding said sgRNA-R;
wherein the nucleotide sequences of DNA molecules A are shown in SEQ ID NO:26, and the nucleotide sequences of DNA molecules B are shown in SEQ ID NO:27.

13. The method according to claim 1, which is characterized in that, said construction of targeting vector in step 3) include the construction of targeting vector with site-specific cleavage and the targeting vector to insert the gene.

14. The method according to claim 13, which is characterized in that, the steps of construction of targeting vector to insert the gene aimed at site-specific cleavage system are as follows: 1) design of the 5′ terminal homology arm and 3′ terminal homology arm with their gene knocked out and the corresponding universal primers; 2) obtain the targeting vector by leading said homology arms, universal primers, marker gene and/or genes to be inserted into the carrier.

15. The method according to claim 14, which is characterized in that, said 5′ terminal homology arm and 3′ terminal homology arm in the step 1) on construction of targeting vector to insert the gene, wherein the nucleotide sequences of the 5′ terminal homology arm are shown in SEQ ID NO:28, and the nucleotide sequences of corresponding universal primers are shown in SEQ ID NO:29; the nucleotide sequences of the 3′ terminal homology arm are shown in SEQ ID NO:30, and the nucleotide sequences of corresponding universal primers are shown in SEQ ID NO:31.

16. The method according to claim 14, which is characterized in that, the sequences of targeting vector to insert the gene constructed for site-specific cleavage system include above mentioned the sequences of 5′ terminal homology, the universal primers sequences of 5′ terminal homology, the gene sequences to be inserted, the universal primers sequences of 3′ terminal homology, the sequences of 3′ terminal homology.

17. The method according to claim 16, which is characterized in that, the nucleotide sequences of targeting vector to insert the gene constructed for site-specific cleavage system are shown in SEQ ID NO:32.

18. The method according to claim 1, which is characterized in that, the nucleotide sequences of PCR amplified primers used in PCR amplification to identify insertion results in step 4) are shown in SEQ ID NO:33, SEQ ID NO:34, SEQ ID NO:35, SEQ ID NO:36, SEQ ID NO:37, SEQ ID NO:38.

19. The application of the method of claim 1 in targeted modification of porcine H11 gene.

20. The application of the method of claim 1 in the construction of porcine H11 gene mutation library.


SEQUENCE LISTING (45 sequences)

SEQ ID NO: 1 - DNA, 18 bp, Artificial sequence
  Sus scrofa
ttcttatgttcctggaag

SEQ ID NO: 2 - DNA, 18 bp, Artificial sequence
  Sus scrofa
tcttatgttcctggaagt

SEQ ID NO: 3 - DNA, 18 bp, Artificial sequence
  Sus scrofa
cttatgttcctggaagtt

SEQ ID NO: 4 - DNA, 18 bp, Artificial sequence
  Sus scrofa
gacccaaaatatccgatg

SEQ ID NO: 5 - DNA, 18 bp, Artificial sequence
  Sus scrofa
gagacccaaaatatccga

SEQ ID NO: 6 - DNA, 23 bp, Artificial sequence
  Sus scrofa
gttcctggaagtttagatcaggg

SEQ ID NO: 7 - DNA, 23 bp, Artificial sequence
  Sus scrofa
agatcagggtgggcagctctggg

SEQ ID NO: 8 - DNA, 23 bp, Artificial sequence
  Sus scrofa
agatcagggtgggcagctctggg

SEQ ID NO: 9 - DNA, 23 bp, Artificial sequence
  Sus scrofa
ttccaggaacataagaaagtagg

SEQ ID NO: 10 - protein, 646 aa, Artificial sequence
  synthesized
LTPEQVVAIASNGGGKQALETVQRLLPVLCQAHGLTPEQVVAIASNGGGKQALETVQRLLPVLCQAHGLTPEQVVAIASHDGGKQALETVQRLLPVLCQAHGLTPEQVVAIASNGGGKQALETVQRLLPVLCQAHGLTPEQVVAIASNGGGKQALETVQRLLPVLCQAHGLTPEQVVAIASNIGGKQALETVQRLLPVLCQAHGLTPEQVVAIASNGGGKQALETVQRLLPVLCQAHGLTPEQVVAIASNNGGKQALETVQRLLPVLCQAHGLTPEQVVAIASNGGGKQALETVQRLLPVLCQAHGLTPEQVVAIASNGGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASNGGGKQALETVQRLLPVLCQAHGLTPEQVVAIASNNGGKQALETVQRLLPVLCQAHGLTPEQVVAIASNNGGKQALETVQRLLPVLCQAHGLTPEQVVAIASNIGGKQALETVQRLLPVLCQAHGLTPEQVVAIASNIGGKQALETVQRLLPVLCQAHGLTPEQVVAIASNNGGKQALETVQRLLPVLCQAHGLTPEQVVAIASNGGGKQALETVQRLLPVLCQAHG

SEQ ID NO: 11 - protein, 646 aa, Artificial sequence
  synthesized
LTPEQVVAIASNNGGKQALETVQRLLPVLCQAHGLTPEQVVAIASNGGGKQALETVQRLLPVLCQAHGLTPEQVVAIASNIGGKQALETVQRLLPVLCQAHGLTPEQVVAIASNNGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASNGGGKQALETVQRLLPVLCQAHGLTPEQVVAIASNIGGKQALETVQRLLPVLCQAHGLTPEQVVAIASNGGGKQALETVQRLLPVLCQAHGLTPEQVVAIASNIGGKQALETVQRLLPVLCQAHGLTPEQVVAIASNIGGKQALETVQRLLPVLCQAHGLTPEQVVAIASNIGGKQALETVQRLLPVLCQAHGLTPEQVVAIASNIGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASNIGGKQALETVQRLLPVLCQAHGLTPEQVVAIASNNGGKQALETVQRLLPVLCQAHGLTPEQVVAIASNIGGKQALETVQRLLPVLCQAHG

SEQ ID NO: 12 - protein, 646 aa, Artificial sequence
  synthesized
LTPEQVVAIASNGGGKQALETVQRLLPVLCQAHGLTPEQVVAIASHDGGKQALETVQRLLPVLCQAHGLTPEQVVAIASNGGGKQALETVQRLLPVLCQAHGLTPEQVVAIASNGGGKQALETVQRLLPVLCQAHGLTPEQVVAIASNIGGKQALETVQRLLPVLCQAHGLTPEQVVAIASNGGGKQALETVQRLLPVLCQAHGLTPEQVVAIASNNGGKQALETVQRLLPVLCQAHGLTPEQVVAIASNGGGKQALETVQRLLPVLCQAHGLTPEQVVAIASNGGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASNGGGKQALETVQRLLPVLCQAHGLTPEQVVAIASNNGGKQALETVQRLLPVLCQAHGLTPEQVVAIASNNGGKQALETVQRLLPVLCQAHGLTPEQVVAIASNIGGKQALETVQRLLPVLCQAHGLTPEQVVAIASNIGGKQALETVQRLLPVLCQAHGLTPEQVVAIASNNGGKQALETVQRLLPVLCQAHGLTPEQVVAIASNGGGKQALETVQRLLPVLCQAHGLTPEQVVAIASNGGGKQALETVQRLLPVLCQAHG

SEQ ID NO: 13 - protein, 646 aa, Artificial sequence
  synthesized
LTPEQVVAIASHDGGKQALETVQRLLPVLCQAHGLTPEQVVAIASNGGGKQALETVQRLLPVLCQAHGLTPEQVVAIASNGGGKQALETVQRLLPVLCQAHGLTPEQVVAIASNIGGKQALETVQRLLPVLCQAHGLTPEQVVAIASNGGGKQALETVQRLLPVLCQAHGLTPEQVVAIASNNGGKQALETVQRLLPVLCQAHGLTPEQVVAIASNGGGKQALETVQRLLPVLCQAHGLTPEQVVAIASNGGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASNGGGKQALETVQRLLPVLCQAHGLTPEQVVAIASNNGGKQALETVQRLLPVLCQAHGLTPEQVVAIASNNGGKQALETVQRLLPVLCQAHGLTPEQVVAIASNIGGKQALETVQRLLPVLCQAHGLTPEQVVAIASNIGGKQALETVQRLLPVLCQAHGLTPEQVVAIASNNGGKQALETVQRLLPVLCQAHGLTPEQVVAIASNGGGKQALETVQRLLPVLCQAHGLTPEQVVAIASNGGGKQALETVQRLLPVLCQAHGLTPEQVVAIASNGGGKQALETVQRLLPVLCQAHG

SEQ ID NO: 14 - protein, 646 aa, Artificial sequence
  synthesized
LTPEQVVAIASNIGGKQALETVQRLLPVLCQAHGLTPEQVVAIASNNGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASNGGGKQALETVQRLLPVLCQAHGLTPEQVVAIASNIGGKQALETVQRLLPVLCQAHGLTPEQVVAIASNGGGKQALETVQRLLPVLCQAHGLTPEQVVAIASNIGGKQALETVQRLLPVLCQAHGLTPEQVVAIASNIGGKQALETVQRLLPVLCQAHGLTPEQVVAIASNIGGKQALETVQRLLPVLCQAHGLTPEQVVAIASNIGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASNIGGKQALETVQRLLPVLCQAHGLTPEQVVAIASNNGGKQALETVQRLLPVLCQAHGLTPEQVVAIASNIGGKQALETVQRLLPVLCQAHGLTPEQVVAIASNNGGKQALETVQRLLPVLCQAHGLTPEQVVAIASHDGGKQALETVQRLLPVLCQAHG

SEQ ID NO: 15 - RNA, 20 nt, Artificial sequence
  synthesized
ugaucuaaacuuccaggaac

SEQ ID NO: 16 - RNA, 20 nt, Artificial sequence
  synthesized
agagcugcccacccugaucu

SEQ ID NO: 17 - RNA, 20 nt, Artificial sequence
  Sus scrofa
agagcugcccacccugaucu

SEQ ID NO: 18 - RNA, 20 nt, Artificial sequence
  Sus scrofa
acuuucuuauguuccuggaa

SEQ ID NO: 19 - DNA, 1938 bp, Artificial sequence
  synthesized
ctgaccccagagcaggtcgtggccattgcctcgaatggagggggcaaacaggcgttggaaaccgtacaacgattgctgccggtgctttgtcaggcacacggcctgaccccagagcaggtcgtggccattgcctcgaatggagggggcaaacaggcgttggaaaccgtacaacgattgctgccggtgctttgtcaggcacacggcctgaccccagagcaggtcgtggcgatcgcaagccacgacggaggaaagcaagccttggaaacagtacagaggctgttgcctgtgctttgtcaggcacacggcctgaccccagagcaggtcgtggccattgcctcgaatggagggggcaaacaggcgttggaaaccgtacaacgattgctgccggtgctttgtcaggcacacggcctgaccccagagcaggtcgtggccattgcctcgaatggagggggcaaacaggcgttggaaaccgtacaacgattgctgccggtgctttgtcaggcacacggcctcactccggaacaagtggtcgcaatcgcctccaacattggcgggaaacaggcactcgagactgtccagcgcctgcttcccgtgctgtgccaagcgcacggtctgaccccagagcaggtcgtggccattgcctcgaatggagggggcaaacaggcgttggaaaccgtacaacgattgctgccggtgctttgtcaggcacacggcctcactccggaacaagtggtcgcaatcgcgagcaataacggcggaaaacaggctttggaaacggtgcagaggctccttccagtgctgtgccaagcgcacggtctgaccccagagcaggtcgtggccattgcctcgaatggagggggcaaacaggcgttggaaaccgtacaacgattgctgccggtgctttgtcaggcacacggcctgaccccagagcaggtcgtggccattgcctcgaatggagggggcaaacaggcgttggaaaccgtacaacgattgctgccggtgctttgtcaggcacacggcctgaccccagagcaggtcgtggcgatcgcaagccacgacggaggaaagcaagccttggaaacagtacagaggctgttgcctgtgctttgtcaggcacacggcctgaccccagagcaggtcgtggcgatcgcaagccacgacggaggaaagcaagccttggaaacagtacagaggctgttgcctgtgctttgtcaggcacacggcctgaccccagagcaggtcgtggccattgcctcgaatggagggggcaaacaggcgttggaaaccgtacaacgattgctgccggtgctttgtcaggcacacggcctcactccggaacaagtggtcgcaatcgcgagcaataacggcggaaaacaggctttggaaacggtgcagaggctccttccagtgctgtgccaagcgcacggtctcactccggaacaagtggtcgcaatcgcgagcaataacggcggaaaacaggctttggaaacggtgcagaggctccttccagtgctgtgccaagcgcacggtctcactccggaacaagtggtcgcaatcgcctccaacattggcgggaaacaggcactcgagactgtccagcgcctgcttcccgtgctgtgccaagcgcacggtctcactccggaacaagtggtcgcaatcgcctccaacattggcgggaaacaggcactcgagactgtccagcgcctgcttcccgtgctgtgccaagcgcacggtctcactccggaacaagtggtcgcaatcgcgagcaataacggcggaaaacaggctttggaaacggtgcagaggctccttccagtgctgtgccaagcgcacggtctgaccccagagcaggtcgtggccattgcctcgaatggagggggcaaacaggcgttggaaaccgtacaacgattgctgccggtgctttgtcaggcacacggc

SEQ ID NO: 20 - DNA, 1938 bp, Artificial sequence
  synthesized
ctgaccccagagcaggtcgtggccattgcctcgaatggagggggcaaacaggcgttggaaaccgtacaacgattgctgccggtgctttgtcaggcacacggcctgaccccagagcaggtcgtggcgatcgcaagccacgacggaggaaagcaagccttggaaacagtacagaggctgttgcctgtgctttgtcaggcacacggcctgaccccagagcaggtcgtggccattgcctcgaatggagggggcaaacaggcgttggaaaccgtacaacgattgctgccggtgctttgtcaggcacacggcctgaccccagagcaggtcgtggccattgcctcgaatggagggggcaaacaggcgttggaaaccgtacaacgattgctgccggtgctttgtcaggcacacggcctcactccggaacaagtggtcgcaatcgcctccaacattggcgggaaacaggcactcgagactgtccagcgcctgcttcccgtgctgtgccaagcgcacggtctgaccccagagcaggtcgtggccattgcctcgaatggagggggcaaacaggcgttggaaaccgtacaacgattgctgccggtgctttgtcaggcacacggcctcactccggaacaagtggtcgcaatcgcgagcaataacggcggaaaacaggctttggaaacggtgcagaggctccttccagtgctgtgccaagcgcacggtctgaccccagagcaggtcgtggccattgcctcgaatggagggggcaaacaggcgttggaaaccgtacaacgattgctgccggtgctttgtcaggcacacggcctgaccccagagcaggtcgtggccattgcctcgaatggagggggcaaacaggcgttggaaaccgtacaacgattgctgccggtgctttgtcaggcacacggcctgaccccagagcaggtcgtggcgatcgcaagccacgacggaggaaagcaagccttggaaacagtacagaggctgttgcctgtgctttgtcaggcacacggcctgaccccagagcaggtcgtggcgatcgcaagccacgacggaggaaagcaagccttggaaacagtacagaggctgttgcctgtgctttgtcaggcacacggcctgaccccagagcaggtcgtggccattgcctcgaatggagggggcaaacaggcgttggaaaccgtacaacgattgctgccggtgctttgtcaggcacacggcctcactccggaacaagtggtcgcaatcgcgagcaataacggcggaaaacaggctttggaaacggtgcagaggctccttccagtgctgtgccaagcgcacggtctcactccggaacaagtggtcgcaatcgcgagcaataacggcggaaaacaggctttggaaacggtgcagaggctccttccagtgctgtgccaagcgcacggtctcactccggaacaagtggtcgcaatcgcctccaacattggcgggaaacaggcactcgagactgtccagcgcctgcttcccgtgctgtgccaagcgcacggtctcactccggaacaagtggtcgcaatcgcctccaacattggcgggaaacaggcactcgagactgtccagcgcctgcttcccgtgctgtgccaagcgcacggtctcactccggaacaagtggtcgcaatcgcgagcaataacggcggaaaacaggctttggaaacggtgcagaggctccttccagtgctgtgccaagcgcacggtctgaccccagagcaggtcgtggccattgcctcgaatggagggggcaaacaggcgttggaaaccgtacaacgattgctgccggtgctttgtcaggcacacggcctgaccccagagcaggtcgtggccattgcctcgaatggagggggcaaacaggcgttggaaaccgtacaacgattgctgccggtgctttgtcaggcacacggc

SEQ ID NO: 21 - DNA, 1938 bp, Artificial sequence
  synthesized
ctgaccccagagcaggtcgtggcgatcgcaagccacgacggaggaaagcaagccttggaaacagtacagaggctgttgcctgtgctttgtcaggcacacggcctgaccccagagcaggtcgtggccattgcctcgaatggagggggcaaacaggcgttggaaaccgtacaacgattgctgccggtgctttgtcaggcacacggcctgaccccagagcaggtcgtggccattgcctcgaatggagggggcaaacaggcgttggaaaccgtacaacgattgctgccggtgctttgtcaggcacacggcctcactccggaacaagtggtcgcaatcgcctccaacattggcgggaaacaggcactcgagactgtccagcgcctgcttcccgtgctgtgccaagcgcacggtctgaccccagagcaggtcgtggccattgcctcgaatggagggggcaaacaggcgttggaaaccgtacaacgattgctgccggtgctttgtcaggcacacggcctcactccggaacaagtggtcgcaatcgcgagcaataacggcggaaaacaggctttggaaacggtgcagaggctccttccagtgctgtgccaagcgcacggtctgaccccagagcaggtcgtggccattgcctcgaatggagggggcaaacaggcgttggaaaccgtacaacgattgctgccggtgctttgtcaggcacacggcctgaccccagagcaggtcgtggccattgcctcgaatggagggggcaaacaggcgttggaaaccgtacaacgattgctgccggtgctttgtcaggcacacggcctgaccccagagcaggtcgtggcgatcgcaagccacgacggaggaaagcaagccttggaaacagtacagaggctgttgcctgtgctttgtcaggcacacggcctgaccccagagcaggtcgtggcgatcgcaagccacgacggaggaaagcaagccttggaaacagtacagaggctgttgcctgtgctttgtcaggcacacggcctgaccccagagcaggtcgtggccattgcctcgaatggagggggcaaacaggcgttggaaaccgtacaacgattgctgccggtgctttgtcaggcacacggcctcactccggaacaagtggtcgcaatcgcgagcaataacggcggaaaacaggctttggaaacggtgcagaggctccttccagtgctgtgccaagcgcacggtctcactccggaacaagtggtcgcaatcgcgagcaataacggcggaaaacaggctttggaaacggtgcagaggctccttccagtgctgtgccaagcgcacggtctcactccggaacaagtggtcgcaatcgcctccaacattggcgggaaacaggcactcgagactgtccagcgcctgcttcccgtgctgtgccaagcgcacggtctcactccggaacaagtggtcgcaatcgcctccaacattggcgggaaacaggcactcgagactgtccagcgcctgcttcccgtgctgtgccaagcgcacggtctcactccggaacaagtggtcgcaatcgcgagcaataacggcggaaaacaggctttggaaacggtgcagaggctccttccagtgctgtgccaagcgcacggtctgaccccagagcaggtcgtggccattgcctcgaatggagggggcaaacaggcgttggaaaccgtacaacgattgctgccggtgctttgtcaggcacacggcctgaccccagagcaggtcgtggccattgcctcgaatggagggggcaaacaggcgttggaaaccgtacaacgattgctgccggtgctttgtcaggcacacggcctgaccccagagcaggtcgtggccattgcctcgaatggagggggcaaacaggcgttggaaaccgtacaacgattgctgccggtgctttgtcaggcacacggc

SEQ ID NO: 22 - DNA, 1938 bp, Artificial sequence
  synthesized
ctcactccggaacaagtggtcgcaatcgcgagcaataacggcggaaaacaggctttggaaacggtgcagaggctccttccagtgctgtgccaagcgcacggtctgaccccagagcaggtcgtggccattgcctcgaatggagggggcaaacaggcgttggaaaccgtacaacgattgctgccggtgctttgtcaggcacacggcctcactccggaacaagtggtcgcaatcgcctccaacattggcgggaaacaggcactcgagactgtccagcgcctgcttcccgtgctgtgccaagcgcacggtctcactccggaacaagtggtcgcaatcgcgagcaataacggcggaaaacaggctttggaaacggtgcagaggctccttccagtgctgtgccaagcgcacggtctgaccccagagcaggtcgtggcgatcgcaagccacgacggaggaaagcaagccttggaaacagtacagaggctgttgcctgtgctttgtcaggcacacggcctgaccccagagcaggtcgtggcgatcgcaagccacgacggaggaaagcaagccttggaaacagtacagaggctgttgcctgtgctttgtcaggcacacggcctgaccccagagcaggtcgtggccattgcctcgaatggagggggcaaacaggcgttggaaaccgtacaacgattgctgccggtgctttgtcaggcacacggcctcactccggaacaagtggtcgcaatcgcctccaacattggcgggaaacaggcactcgagactgtccagcgcctgcttcccgtgctgtgccaagcgcacggtctgaccccagagcaggtcgtggccattgcctcgaatggagggggcaaacaggcgttggaaaccgtacaacgattgctgccggtgctttgtcaggcacacggcctcactccggaacaagtggtcgcaatcgcctccaacattggcgggaaacaggcactcgagactgtccagcgcctgcttcccgtgctgtgccaagcgcacggtctcactccggaacaagtggtcgcaatcgcctccaacattggcgggaaacaggcactcgagactgtccagcgcctgcttcccgtgctgtgccaagcgcacggtctcactccggaacaagtggtcgcaatcgcctccaacattggcgggaaacaggcactcgagactgtccagcgcctgcttcccgtgctgtgccaagcgcacggtctcactccggaacaagtggtcgcaatcgcctccaacattggcgggaaacaggcactcgagactgtccagcgcctgcttcccgtgctgtgccaagcgcacggtctgaccccagagcaggtcgtggcgatcgcaagccacgacggaggaaagcaagccttggaaacagtacagaggctgttgcctgtgctttgtcaggcacacggcctgaccccagagcaggtcgtggcgatcgcaagccacgacggaggaaagcaagccttggaaacagtacagaggctgttgcctgtgctttgtcaggcacacggcctgaccccagagcaggtcgtggcgatcgcaagccacgacggaggaaagcaagccttggaaacagtacagaggctgttgcctgtgctttgtcaggcacacggcctcactccggaacaagtggtcgcaatcgcctccaacattggcgggaaacaggcactcgagactgtccagcgcctgcttcccgtgctgtgccaagcgcacggtctcactccggaacaagtggtcgcaatcgcgagcaataacggcggaaaacaggctttggaaacggtgcagaggctccttccagtgctgtgccaagcgcacggtctcactccggaacaagtggtcgcaatcgcctccaacattggcgggaaacaggcactcgagactgtccagcgcctgcttcccgtgctgtgccaagcgcacggt

SEQ ID NO: 23 - DNA, 1938 bp, Artificial sequence
  synthesized
ctcactccggaacaagtggtcgcaatcgcctccaacattggcgggaaacaggcactcgagactgtccagcgcctgcttcccgtgctgtgccaagcgcacggtctcactccggaacaagtggtcgcaatcgcgagcaataacggcggaaaacaggctttggaaacggtgcagaggctccttccagtgctgtgccaagcgcacggtctgaccccagagcaggtcgtggcgatcgcaagccacgacggaggaaagcaagccttggaaacagtacagaggctgttgcctgtgctttgtcaggcacacggcctgaccccagagcaggtcgtggcgatcgcaagccacgacggaggaaagcaagccttggaaacagtacagaggctgttgcctgtgctttgtcaggcacacggcctgaccccagagcaggtcgtggccattgcctcgaatggagggggcaaacaggcgttggaaaccgtacaacgattgctgccggtgctttgtcaggcacacggcctcactccggaacaagtggtcgcaatcgcctccaacattggcgggaaacaggcactcgagactgtccagcgcctgcttcccgtgctgtgccaagcgcacggtctgaccccagagcaggtcgtggccattgcctcgaatggagggggcaaacaggcgttggaaaccgtacaacgattgctgccggtgctttgtcaggcacacggcctcactccggaacaagtggtcgcaatcgcctccaacattggcgggaaacaggcactcgagactgtccagcgcctgcttcccgtgctgtgccaagcgcacggtctcactccggaacaagtggtcgcaatcgcctccaacattggcgggaaacaggcactcgagactgtccagcgcctgcttcccgtgctgtgccaagcgcacggtctcactccggaacaagtggtcgcaatcgcctccaacattggcgggaaacaggcactcgagactgtccagcgcctgcttcccgtgctgtgccaagcgcacggtctcactccggaacaagtggtcgcaatcgcctccaacattggcgggaaacaggcactcgagactgtccagcgcctgcttcccgtgctgtgccaagcgcacggtctgaccccagagcaggtcgtggcgatcgcaagccacgacggaggaaagcaagccttggaaacagtacagaggctgttgcctgtgctttgtcaggcacacggcctgaccccagagcaggtcgtggcgatcgcaagccacgacggaggaaagcaagccttggaaacagtacagaggctgttgcctgtgctttgtcaggcacacggcctgaccccagagcaggtcgtggcgatcgcaagccacgacggaggaaagcaagccttggaaacagtacagaggctgttgcctgtgctttgtcaggcacacggcctcactccggaacaagtggtcgcaatcgcctccaacattggcgggaaacaggcactcgagactgtccagcgcctgcttcccgtgctgtgccaagcgcacggtctcactccggaacaagtggtcgcaatcgcgagcaataacggcggaaaacaggctttggaaacggtgcagaggctccttccagtgctgtgccaagcgcacggtctcactccggaacaagtggtcgcaatcgcctccaacattggcgggaaacaggcactcgagactgtccagcgcctgcttcccgtgctgtgccaagcgcacggtctcactccggaacaagtggtcgcaatcgcgagcaataacggcggaaaacaggctttggaaacggtgcagaggctccttccagtgctgtgccaagcgcacggtctgaccccagagcaggtcgtggcgatcgcaagccacgacggaggaaagcaagccttggaaacagtacagaggctgttgcctgtgctttgtcaggcacacggc

SEQ ID NO: 24 - DNA, 20 bp, Artificial sequence
  synthesized
gttcctggaagtttagatca

SEQ ID NO: 25 - DNA, 20 bp, Artificial sequence
  synthesized
agatcagggtgggcagctct

SEQ ID NO: 26 - DNA, 20 bp, Artificial sequence
  synthesized
agatcagggtgggcagctct

SEQ ID NO: 27 - DNA, 20 bp, Artificial sequence
  synthesized
ttccaggaacataagaaagt

SEQ ID NO: 28 - DNA, 808 bp, Artificial sequence
  Sus scrofa
cattgagccacgaacagaactccctcttaccaacttattactactaacttcccaagtactggctgctcagctgcttccttgggcatgggggagggagcactattttttcctctcctgacttcatcctcttccttttaatttccataaggttccctgtggccctgtgcttttttattttgaggccttgcacatccttctggccctgattgcttctcaactcatcttgtgcctgctggacttccaccgttgtttcatgtatctcgttagctgagatagcacttcctcctgcccttaccctttatctggctcttagctcctgaaaactgcattattagcttcctcttttgcctctactcttactcaaccaaaattgttttaagatctgtggatctagcttctgctgtgctattcttaggaacacttttatttcctcttagctccatctcaccagttattggctaatggctttgcttggtacctacatctgtacatttctttcgtactagcttctagactgaaaaaggactgttggttcaacatgaaagggaaggaggtaaaagaggacacacaggaaagatggattgggattcaggtctctgctgttgttacttgagattgctttctagattctacttgtggaaacaaaaagcctttgcgagaattctaaactggagtatttctgtaattgaggagtcttgctcagcaaatcccacttaggggactaatgaagtaccaggaagagacagaccatgctcaatccacaaagccaggttttactgaaatgtgacctactttcttatgcgatcgc

SEQ ID NO: 29 - DNA, 63 bp, Artificial sequence
  synthesized
ctgccgaaagagtaatgttggccgagataggagaagacgatgatatcacgctacgacggaaac

SEQ ID NO: 30 - DNA, 800 bp, Artificial sequence
  Sus scrofa
ttttataggctacactgttaacactcaggctgttttctaccgtttagtcaaaatatagtcaccttgcctgcttcacctgtccatcagagaatggcctcattaattgactctctagtatgaagtcaaagtagctttggtggccctaaatggacaagtatcaagagactgggtgaattgaggagcttgagactgtcacctcagatcgaaaagactgaaaaatcacctcagatcaaaaagactgaaaaatcttcagtctggaaaggggactcaaaaccataattagagtattctggtagaatccttttctccactgttattcatacagttaaggtgaataactaaaagtaattgtgagctgaggagtaagatacaacacacaaggaatcagttaacagagtctcgagtgaaattataaatggaaagaattatgacttgaatcataactctgaggccccattttccctaacaacttttgtcccaataaacgtgggtatttgtttgggagaaactatcatatacatgattacccagtaaacagactgtttactaagtgggtttaattttagaaattgcgcgctgcaatctggtattaaccatacaactacctacctatagggtcagcccagcctgaactatcccattggggtctttattaaggctcaagaaacggccatagcttcttcctttaaaatgagtgtttatttctatgagctttaaagaaaaaaacagataatttccctcaacctactgaagaggaagggattcaggaagaaataaacacaacaatgccattcacttca

SEQ ID NO: 31 - DNA, 66 bp, Artificial sequence
  synthesized
ttcccgaggctgagttagttggtccagccagtgattgagttgcgtgcggagggcttcttatcttag

SEQ ID NO: 32 - DNA, 10301 bp, Artificial sequence
  synthesized
ctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctcgaaattaaccctcactaaagggaacaaaagctggagctacttaagggcgcgcccattgagccacgaacagaactccctcttaccaacttattactactaacttcccaagtactggctgctcagctgcttccttgggcatgggggagggagcactattttttcctctcctgacttcatcctcttccttttaatttccataaggttccctgtggccctgtgcttttttattttgaggccttgcacatccttctggccctgattgcttctcaactcatcttgtgcctgctggacttccaccgttgtttcatgtatctcgttagctgagatagcacttcctcctgcccttaccctttatctggctcttagctcctgaaaactgcattattagcttcctcttttgcctctactcttactcaaccaaaattgttttaagatctgtggatctagcttctgctgtgctattcttaggaacacttttatttcctcttagctccatctcaccagttattggctaatggctttgcttggtacctacatctgtacatttctttcgtactagcttctagactgaaaaaggactgttggttcaacatgaaagggaaggaggtaaaagaggacacacaggaaagatggattgggattcaggtctctgctgttgttacttgagattgctttctagattctacttgtggaaacaaaaagcctttgcgagaattctaaactggagtatttctgtaattgaggagtcttgctcagcaaatcccacttaggggactaatgaagtaccaggaagagacagaccatgctcaatccacaaagccaggttttactgaaatgtgacctactttcttatgcgatcgcctgccgaaagagtaatgttggccgagataggagaagacgatgatatcacgctacgacggaaacagtactatggcctcctccgaggacgtcatcaaggagttcatgcgcttcaaggtgcgcatggagggctccgtgaacggccacgagttcgagatcgagggcgagggcgagggccgcccctacgagggcacccagaccgccaagctgaaggtgaccaagggcggccccctgcccttcgcctgggacatcctgtcccctcagttccagtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctccaccgagcggatgtaccccgaggacggcgccctgaagggcgagatcaagatgaggctgaagctgaaggacggcggccactacgacgccgaggtcaagaccacctacatggccaagaagcccgtgcagctgcccggcgcctacaagaccgacatcaagctggacatcacctcccacaacgaggactacaccatcgtggaacagtacgagcgcgccgagggccgccactccaccggcgcctaagaatgcaattgttgttgttaacttgtttattgcagcttataatggttacaaataaagcaatagcatcacaaatttcacaaataaagcatttttttcactgcattctagttgtggtttgtccaaactcatcaatgtatcttattaattaaacgcggtggcggccgcattaccctgttatccctagaattcgatgctgaagttcctatagtttctagagtataggaacttcggtcataacttcgtatagcatacattatacgaagttattccggataagatacattgatgagtttggacaaaccacaactagaatgcagtgaaaaaaatgctttatttgtgaaatttgtgatgctattgctttatttgtaaccattataagctgcaataaacaagttggggtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatgctagcttatcatcgtgtttttcaaaggaaaaccacgtccccgtggttcggggggcctagacgtttttttaacctcgactaaacacatgtaaagcatgtgcaccgaggccccagatcagatcccatacaatggggtaccttctgggcatccttcagccccttgttgaatacgcttgaggagagccatttgactctttccacaactatccaactcacaacgtggcactggggttgtgccgcctttgcaggtgtatcttatacacgtggcttttggccgcagaggcacctgtcgccaggtggggggttccgctgcctgcaaagggtcgctacagacgttgtttgtcttcaagaagcttccagaggaactgcttccttcacgacattcaacagaccttgcattcctttggcgagaggggaaagacccctaggaatgctcgtcaagaagacagggccaggtttccgggccctcacattgccaaaagacggcaatatggtggaaaataacatatagacaaacgcacaccggccttattccaagcggcttcggccagtaacgttagggggggggggggagaggggcggaattggatccgatatcttacttgtacagctcgtccatgccgagagtgatcccggcggcggtcacgaactccagcaggaccatgtgatcgcgcttctcgttggggtctttgctcagggcggactgggtgctcaggtagtggttgtcgggcagcagcacggggccgtcgccgatgggggtgttctgctggtagtggtcggcgagctgcacgctgccgtcctcgatgttgtggcggatcttgaagttcaccttgatgccgttcttctgcttgtcggccatgatatagacgttgtggctgttgtagttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcggctgaagcactgcacgccgtaggtcagggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccatcttaaggatctgacggttcactaaaccagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcggaactccatatatgggctatgaactaatgaccccgtaattgagatctgaagttcctatagtttctagagtataggaacttcggtcataacttcgtatagcatacattatacgaagttatacgcgtttcccgaggctgagttagttggtccagccagtgattgagttgcgtgcggagggcttcttatcttagttttataggctacactgttaacactcaggctgttttctaccgtttagtcaaaatatagtcaccttgcctgcttcacctgtccatcagagaatggcctcattaattgactctctagtatgaagtcaaagtagctttggtggccctaaatggacaagtatcaagagactgggtgaattgaggagcttgagactgtcacctcagatcgaaaagactgaaaaatcacctcagatcaaaaagactgaaaaatcttcagtctggaaaggggactcaaaaccataattagagtattctggtagaatccttttctccactgttattcatacagttaaggtgaataactaaaagtaattgtgagctgaggagtaagatacaacacacaaggaatcagttaacagagtctcgagtgaaattataaatggaaagaattatgacttgaatcataactctgaggccccattttccctaacaacttttgtcccaataaacgtgggtatttgtttgggagaaactatcatatacatgattacccagtaaacagactgtttactaagtgggtttaattttagaaattgcgcgctgcaatctggtattaaccatacaactacctacctatagggtcagcccagcctgaactatcccattggggtctttattaaggctcaagaaacggccatagcttcttcctttaaaatgagtgtttatttctatgagctttaaagaaaaaaacagataatttccctcaacctactgaagaggaagggattcaggaagaaataaacacaacaatgccattcacttcaggccggcctctagaatgcatgtttaaacaggccgcgggaattcgattatcgaattctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctgcaggtcctcgccatggatcctgatgatgttgttgattcttctaaatcttttgtgatggaaaacttttcttcgtaccacgggactaaacctggttatgtagattccattcaaaaaggtatacaaaagccaaaatctggtacacaaggaaattatgacgatgattggaaagggttttatagtaccgacaataaatacgacgctgcgggatactctgtagataatgaaaacccgctctctggaaaagctggaggcgtggtcaaagtgacgtatccaggactgacgaaggttctcgcactaaaagtggataatgccgaaactattaagaaagagttaggtttaagtctcactgaaccgttgatggagcaagtcggaacggaagagtttatcaaaaggttcggtgatggtgcttcgcgtgtagtgctcagccttcccttcgctgaggggagttctagcgttgaatatattaataactgggaacaggcgaaagcgttaagcgtagaacttgagattaattttgaaacccgtggaaaacgtggccaagatgcgatgtatgagtatatggctcaagcctgtgcaggaaatcgtgtcaggcgatctctttgtgaaggaaccttacttctgtggtgtgacataattggacaaactacctacagagatttaaagctctaaggtaaatataaaatttttaagtgtataatgtgttaaactactgattctaattgtttgtgtattttagattccaacctatggaactgatgaatgggagcagtggtggaatgcagatcctagagctcgctgatcagcctcgactgtgccttctagttgccagccatctattgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgagggggggcccggtacccaattcgcc

SEQ ID NO: 33 - DNA, 21 bp, Artificial sequence
  synthesized
ctcagtcccaggctttacatc

SEQ ID NO: 34 - DNA, 21 bp, Artificial sequence
  synthesized
ccaacattactctttcggcag

SEQ ID NO: 35 - DNA, 20 bp, Artificial sequence
  synthesized
actggctttctgagttaggg

SEQ ID NO: 36 - DNA, 20 bp, Artificial sequence
  synthesized
gtttccgtcgtagcgtgata

SEQ ID NO: 37 - DNA, 20 bp, Artificial sequence
  synthesized
cggagggcttcttatcttag

SEQ ID NO: 38 - DNA, 20 bp, Artificial sequence
  synthesized
gtgtggagctgtttagggac

SEQ ID NO: 39 - DNA, 495 bp, Artificial sequence
  synthesized
aaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggttcctggaagtttagatcagttttagagctagaaatagcaagttaaaataaggctagtccgttatcaacttgaaaaagtggcaccgagtcggtgctttttttggatccgcggccgctcgacatgtgagcaaaaggccagcaaaaggccaggaaccgtaaaaaggccgcgttgctggcgtttttccataggctccgcccccctgacgagcatcacaaaaatcgacgctca

SEQ ID NO: 40 - DNA, 495 bp, Artificial sequence
  synthesized
aaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgagatcagggtgggcagctctgttttagagctagaaatagcaagttaaaataaggctagtccgttatcaacttgaaaaagtggcaccgagtcggtgctttttttggatccgcggccgctcgacatgtgagcaaaaggccagcaaaaggccaggaaccgtaaaaaggccgcgttgctggcgtttttccataggctccgcccccctgacgagcatcacaaaaatcgacgctca

SEQ ID NO: 41 - DNA, 8434 bp, Artificial sequence
  synthesized
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgggtcttcgagaagacctgttttagagctagaaatagcaagttaaaataaggctagtccgttatcaacttgaaaaagtggcaccgagtcggtgcttttttgttttagagctagaaatagcaagttaaaataaggctagtccgtttttagcgcgtgcgccaattctgcagacaaatggctctagaggtacccgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggaccggtgccaccatgtacccatacgatgttccagattacgcttcgccgaagaaaaagcgcaaggtcgaagcgtccgacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacagccccaagaagaagagaaaggtggaggccagctaagaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 42 - DNA, 7534 bp, Artificial sequence
  synthesized
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccagatcagggtgggcagctctgttttagagctagaaatagcaagttaaaataaggctagtccgttatcaacttgaaaaagtggcaccgagtcggtgcttttttgttttagagacgatgttccagattacgcttcgccgaagaaaaagcgcaaggtcgaagcgtccgacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacagccccaagaagaagagaaaggtggaggccagctaagaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 43 - DNA, 3876 bp, Artificial sequence
  synthesized
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccagatcagggtgggcagctctgttttagagctagaaatagcaagttaaaataaggctagtccgttatcaacttgaaaaagtggcaccgagtcggtgcttttttgttttagaggttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacagccccaagaagaagagaaaggtggaggccagctaagaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 44 - DNA, 15660 bp, Artificial sequence
  synthesized
ctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctcgaaattaaccctcactaaagggaacaaaagctggagctacttaagggcgcgccatgagatgaactgctctgggatgcctaggtaaatttctctgcatttcagtttctttttaggaaagtcagaactgttccttgcaagatgagttctgagaacagaatgtgttgcagaaagtactggagtctttctaaaaatttatcctatgatatttccaagagacatggtcacccttaagcaaagttatacaagtattcatggtcaattaataccatttgggggggtgtcttttttctagggctgcacccatagcataaggaggttcccaggaggtgtggccgtcagcttatgccacaaccacagaaacaccagatccaagcggcatctgtgacctataccacagctcatagcaacgccagatccttagcccccttgattaaagccagggatcaaacctgcctcctcaaggatgctagtcagactcgtttactctgagccacgacaggaactccaagtaataccatttttaatctggaaaaaaatctaaatatcattaaatccaaccttgttattataaaagaaggtaccccatagcaaaggtagctaattcattcaactaatgtgcagctcattaagggtggagctgggaagtgagatctcctacttagcgtcacatgccaccttgcctaataatgatgtatttgtctatcaaatgcctacaaagacatacagagtctctccctggacagttttcattttattatgtgatcgttactaccccaaagatttctttcttgattttattttgtccctcatattctgtctgtcatccctacattcagatatcagaggtgggggtattggggagggggagatgaggagaggaaaaggattggttggtgcatggccagtcaagttgaagatgactgcaacaatcacgagaaatctctgcaaaactataaaagcttcctggggtgccttctgaaaaagtctgatccaagttgctttattagggcctggaccatttctagaagtagatgaatgcattcctttcattggctaggaggtggggatggggcagagagcatacttctgtttctgcagctgagacctggacatggtgaacctggagtagctacccatatggcatggacaggtccaactgctgccccctcctttgtcccccaagaagccagcaggggcaggatgaaggccaccttggggctgccctgagcctcctgcagtatgcctggcaactactttcttagccatctttaaggcccaatcttgggtaaaatactactcaacccattctttagccaccttctccaaatgcttctagaaagcggcccccacaagtaggttctctgcagcagcacagtgcaaatggaggaacacgacctcagtaattattttgtcactgcaaagtatctacaacctttgctataaaaattaacaccttgctttccctgaaaaatagcccagtcatatccagcattttccagcatccagggcagagtgcttgctcctcccccagtcaacaggactgttcataccgaggaaatgatttgagggttctttaagcatttacgctgttaatgctaaagctttcacgacttctacctgaggggggcttgagggaggggggaggtttatgtccctgcaccgccaggagcctggtctttggtaggaacgcagaggcagccggcgaccttccaccctcagtgtgtccttccccaggagtttagggaagtgaatccctagatccagccaacatttccactcccattttcaagagattaaaaaaaaaaaaaaaaaaaaaaaaaaggaaagcatcggcaggtcagcaaaccagcagttctccatccttgggatcttagcagccgacgaccttaattaaacgcggtggcggccgcattaccctgttatccctagaattcgatgctgaagttcctatagtttctagagtataggaacttcggtcataacttcgtatagcatacattatacgaagttattccggataagatacattgatgagtttggacaaaccacaactagaatgcagtgaaaaaaatgctttatttgtgaaatttgtgatgctattgctttatttgtaaccattataagctgcaataaacaagttggggtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatgctagcttatcatcgtgtttttcaaaggaaaaccacgtccccgtggttcggggggcctagacgtttttttaacctcgactaaacacatgtaaagcatgtgcaccgaggccccagatcagatcccatacaatggggtaccttctgggcatccttcagccccttgttgaatacgcttgaggagagccatttgactctttccacaactatccaactcacaacgtggcactggggttgtgccgcctttgcaggtgtatcttatacacgtggcttttggccgcagaggcacctgtcgccaggtggggggttccgctgcctgcaaagggtcgctacagacgttgtttgtcttcaagaagcttccagaggaactgcttccttcacgacattcaacagaccttgcattcctttggcgagaggggaaagacccctaggaatgctcgtcaagaagacagggccaggtttccgggccctcacattgccaaaagacggcaatatggtggaaaataacatatagacaaacgcacaccggccttattccaagcggcttcggccagtaacgttagggggggggggggagaggggcggaattggatccgatatcttacttgtacagctcgtccatgccgagagtgatcccggcggcggtcacgaactccagcaggaccatgtgatcgcgcttctcgttggggtctttgctcagggcggactgggtgctcaggtagtggttgtcgggcagcagcacggggccgtcgccgatgggggtgttctgctggtagtggtcggcgagctgcacgctgccgtcctcgatgttgtggcggatcttgaagttcaccttgatgccgttcttctgcttgtcggccatgatatagacgttgtggctgttgtagttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcggctgaagcactgcacgccgtaggtcagggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccatcttaaggatctgacggttcactaaaccagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcggaactccatatatgggctatgaactaatgaccccgtaattgagatctgaagttcctatagtttctagagtataggaacttcggtcataacttcgtatagcatacattatacgaagttatacgcgttagaatactcaagctatgcatcaagcttggtaccgagctcggatccactagtaacggccgccagtgtgctggaattcgccctttgtccctcttctgttggtagactccactccacttggcggtgatcaccaaccagccagaaatcgctgaggcacttctggaagctggctgtgatcctgagctccgagactttcgaggaaatacccctctacaccttgcctgtgagcagggctgcctggccagtgtgggagtcctgactcagccccgcgggacccagcacctccactccattctgcaggccaccaactacaatggtaagtctggctgccctatgcatcagagggcacgtgacacagacaagggagaggtgggccgacttaaggcaaggtgtaaactcaacacgtggaaggctgagaaaacatgtatgcatcaaggtcttagtaaaacatgtatgcatttgatgccttactaaagtccattcagaacccagagtctgggttcttcaaattcagaagacctcctcccttaaaagaataggtgaaagttctgagaagtgagggtggcaacaagtgcttatattttgttacttttggtcctctaggccacacatgtctgcacttagcctcgatccatggctacctgggcattgtggagctgttggtgtctttgggtgctgatgtcaacgctcaggtgggtgcttcaagcctacagatggagggcattcagccctcaataagatcacatgctcttgctgctagcagaaacctcagactcagccataagcatctcaaattccttttggtttcaggagccctgcaatggccgaaccgccctgcatcttgcggtggacctgcagaatcccgacctggtgtcgctcttgttgaagtgtggggctgatgtcaacagagtcacctaccagggctactccccgtaccagctcacctggggccgcccaagcactcggatacagcagcagctgggccagctgaccctagaaaacctccagatgcttccagagagcgaggatgaggagagctatgacacggagtcagagttcacagaggatgaggtgagtcccaatgaccttgttcacgggtctgcaaaaagcaatgctctcggacccctagagctcctccttttcctgagggtctcaacataatgaggatctcaaattagggagcataagcagtgtcctaagagtaggtttagggggaggattatggtttggggttttcttttgcttttttgctctttttgaaggagaggatccttaaaggaaaacttcagcccaggaagttaattcagattcgggttagagggaacggagtccaagaatacttgcgttatttccagtagcagcccttgccatcaccccagcacctttggcaaagttctggaagtttaacatgcctttctttccccttttagctgccctatgacgactgcgtgcttggaggccagcgcctgacgttatgagctttggaaagtgtctaaaagaccatgtacttgtacatttgtacaaaatcaagagttttatttttctaaaaaaaaagaaaaaaagaaaaaaaaagaaaaaagggtatacttataaccacaccgcacactgcctggcctgaaacattttgctctggtggattagccccgattttgttattcttgtgaactttggaaaggcgccaaggaggatcatcggaatgcagagagaacctcttttaaacggcaccttggtggggcctgggggaaaggttatccctaatttgatgggactcttttatttattgcgcttcttggttgaaccaccatggagtcagtggtggagcccaggtgtatctgggaaatgttagaatcaggtgtgttgttaaacctgtcagtggggtggggttaaaagtcacgacctgtcaaggtttgtgttaccctgctgtaaatactgtacataatgtattttgttggtaattattttggtacttctaagatgtatatttattaaatggatttttacaaacagaattctgatcactgtcttcttcgggcagctgtgggactcctacactgagagtcattcgaaccccaagtggaggtggaggtggagaattgtgtgggagcatttaccacagccaaccacggaactctttcagagaacagcttctcacaccgtctacaccagcctcccggccaggctttgcaggcagccccaggcccagtgcgtgggaggggaggctgttgcaaggtgataggaaacaccagtttcaggcttggggtggcagcaagttggttggcctacagctggaaggctcttcattgtcgcttgctttcatcttcctggtttaaattcagccaggaccttacttctgctttaggaagctttagccaagaggagttagttgtactcatattttgatactagaagttcctgaggacatgggctggggaacaggaccccccactaatgtgttagcaggtgcccacctctgcacctttgtttccctgatgataaaactcggccattggtaaattgcacgagacaatccacgtaacaagcacagcagagtgccaggcacagagtgctaaagaaaaatgcaaactgttgcacaacatcctgtatttcacacgggaaggaacaagaccagaaagaatgtcctggtcaggatcaccttgcaggacaggcaggtggttagcttaacgaatacacgcttgccgtcaggtggctaacatttttgaaatgccatccacctgcaaagcagcctgttttgttcctagtggcagtaccaaattgattataaagactggaagagccttttcatcagccctatctaatgctactgaatgattctagagaagcaagtgatttcacaagaaatcggaaacttgtgaagtttgggtgtagatgctttcaaagtttttatctgtaaaatacatctcttgcctagatagaggagtaaaggaaagtttttggcgctctataaagtacagaatggtatatcaatacacctcatgttctccggctcacacctaggaaaacattactattatattattcctttcctgacctacaaaaaaatgtcaaagagaataagatgttcaccttcctctcagaccaaaaaaaggagccaacacctcatggatctttcatatgcaaagagtatcactatcaactgaactgtctggtcaaagccagctctgtccccctccagcagccacagtcatctagagctgagcactgtggtggcccctctactgtcctgtcttcgctccggtgctccaggcccctcactgaaattctttgtccatcccggggcacctgaacacaggtgttacatgatctaacacgtggatgccagttaggttcagtgcctctgccaacacagagaagagataagagggcttgagggagaaagatccatctctgatcccaaaggacaaaggttggaaattggctccccatttgggaatgatgcccttagtacgtctcagtcagatgtgcctcttttcccctctggaatagtgtcgaatgtacaaatcactccagttgtgtactggtgggctggagaaacaggacaaagggatgtggaccactcttgtgcagccttcttggggtcttgcttttgtgaagaggcatcaccttccatgctcacggagcagaggggccttttctgacccacttgggcccagcctcccagctctccagacttcattcagctttcagagtggattccattgtctgcatacatcaggaggtagctggccccagtttcttaccctctggatttccttccaattcttcatctctttttttttctttttcttttacacctactcgctttcctttgtgccgttggtttgggtggctgaagcctggagttccctaaagaaaaactttagaacccaaacattctagtctagagaaagtcttcctcgaatttctaagcaaacaagaaccaaaattttcaaagaaaacagttcagcagaccaggagcctttaagacactttgatttcctccaagactctaaaggtcctgtcaggacaggtagaagtgaggaagccttgggaaagagggaagtgacagaagagggaaataaaagtcacgtcggcaacttccttgaattgagttccttgattccttctgcctgcctcagcctcaggatgaatttcctttataggtacatgtaccaagtcacttccagaaagaagggtttttctttaaagagggaacaaactccagtctgagcaatttgaagaccttcacgtggggcctggaaataccaaagccggctacttgggggtatttgcattgaaacttcaattgctactggaagtgattaagtggtgagagttagaactgagtcagtgagttgggttcttccttcgccccctttcctcccgattttcatcagctcgctctaggtgtagctgaagtttcattcggcaagaaaggtcagagtggaactccagtcaaaaacgtatctccaaaacattccccaacctctgggacctgggcaggaatatctcgggtcacttccccatcttacagagagcatcacagcaactaaatatcctgtgtgtttgcctcctggagaatcgactctctctcattcattcaatggcatcgtgaagcacctttttttcttcaagccctgtgctaggtgctagagatacaaagaggaagaagtcacggcctctgccccagctcaggtggagtggaagatgagcacatagaagacagggtagtaaggtgaaacccaagacatgatgtgagcacaggtcagagttcccccactcagaaaacagcagaggagaggaatcaagccattatgccctagtaagctctttgcccccccaggccagtactatctattctcttatcctgtgtctggtgaccagtgacctcctttccacaagtggcaggaaacaaagctgtgctcagaataaaactaacttctgggctgggccatccacaacaggctacactgttcacttttgcacccttgtgtgccagattccagagctgagcctgaaagcaatgggtcactagtctttgaattaatgacagatgatgtgcatgacttagtccagagctttccaaacgcgatgtgcacttgactgtgcactcacgtggcctggagatctggttgaaacagtctctgatttagttggtccagggctggcctgacacctgcctctctaacaagttcccaaacaaggcctatgctcctggcccggagaccacactctgaagactattactctgttatttggaataattttgaagaaagtgtaatgttggttaatttaaggccaaagctccctgccatcccactcagtaccactgcaaatctggcaaagcacttctgcagtcactcctgaggatgatccagcatcaggcaacgtgagtctttcagaaaagcaggactggatcctgagaactggcattaaaagtggggctaaggccaaacacctgcctttaaactaggtaagtgtaataagaaaagcagctgacaaaatgcaaggccaaaagcgtaaacacctgaggtccaaggagaggacaaaaatcataaaggaaatgcatttcaaggataatatgctaattaggaagaaaatctggttttaaaatgagcttatatggagttcccttgtggcgtggcatgttaaggatccagcattgtcactgcagagcctagggtcgctgggctgtggcacaggcttgatccctggcctgggagcttctgcatgctatgggcgtggccaaaaaaaaaagaaatccctatgaagttatcaacgacattcttcacagaacaaaatattttaaaatttgtatagaaacacaaaagaccccaaatatccaaggcaatattgaaaaagaaaaattgacctggagaaatcagactcccgacttcagtctgtactacaaagctacagtcatcaaaacagtaaggccctggcataaaaatagaaatatagatcagtggaacaggacagcaatcccagaaataaacccatgcacctatggtcaactagtctatgacaaaggaggcaagaacagtggaggaagacaagggcgaattctgcagatatccatcacactggcggccgggccggcctctagaatgcatgtttaaacaggccgcgggaattcgattatcgaattctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctgcaggtcctcgccatggatcctgatgatgttgttgattcttctaaatcttttgtgatggaaaacttttcttcgtaccacgggactaaacctggttatgtagattccattcaaaaaggtatacaaaagccaaaatctggtacacaaggaaattatgacgatgattggaaagggttttatagtaccgacaataaatacgacgctgcgggatactctgtagataatgaaaacccgctctctggaaaagctggaggcgtggtcaaagtgacgtatccaggactgacgaaggttctcgcactaaaagtggataatgccgaaactattaagaaagagttaggtttaagtctcactgaaccgttgatggagcaagtcggaacggaagagtttatcaaaaggttcggtgatggtgcttcgcgtgtagtgctcagccttcccttcgctgaggggagttctagcgttgaatatattaataactgggaacaggcgaaagcgttaagcgtagaacttgagattaattttgaaacccgtggaaaacgtggccaagatgcgatgtatgagtatatggctcaagcctgtgcaggaaatcgtgtcaggcgatctctttgtgaaggaaccttacttctgtggtgtgacataattggacaaactacctacagagatttaaagctctaaggtaaatataaaatttttaagtgtataatgtgttaaactactgattctaattgtttgtgtattttagattccaacctatggaactgatgaatgggagcagtggtggaatgcagatcctagagctcgctgatcagcctcgactgtgccttctagttgccagccatctattgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgagggggggcccggtacccaattcgcc

SEQ ID NO: 45 - DNA, 10301 bp, Artificial sequence; identical to SEQ ID NO: 32
  synthesized